(19) United States
(12) Patent Application Publication
(10) Pub. No.: US 2009/0179638 A1
(43) Pub. Date: Jul. 16, 2009

(54) ASSAY FOR IDENTIFYING INHIBITORS OF NEURONAL APOPTOTIC PATHWAYS

(75) Inventors: Philip A. Barker, Beaconsfield (CA); James B. Jaquith, Pincourt (CA); Stephen J. Morris, Beaconsfield (CA); Amir Salehi, Pointe-Claire (CA)

Correspondence Address: ROPES & GRAY LLP, PATENT DOCKETING 39/41, ONE INTERNATIONAL PLACE, BOSTON, MA, 02110-2624, US

(73) Assignee: AEGERA THERAPEUTICS INC., Montreal, Quebec (CA)

(21) Appl. No.: 12/278,071

(22) PCT Filed: Feb. 1, 2007
(86) PCT No.: PCT/CA07/00143

§ 371 Date: Nov. 25, 2008

Related U.S. Application Data
(60) Provisional application No. 60/763,887, filed on Feb. 1, 2006.

(51) Int. Cl. G01V 3/00; C07D 513/04; C07F 5/02; G01N 33/68; G01N 33/566

(52) U.S. Cl. 324/307; 548/126; 544/134; 546/268.7; 544/368; 548/110; 436/86; 436/501; 250/281; 250/361 R; 250/305

(57) ABSTRACT

Disclosed is an assay for identifying compounds that modulate a neuronal apoptotic pathway, the assay comprises: a) contacting HSP90 protein with a probe to form a probe: HSP90 complex, the probe being displaceable by a test compound; b) measuring a signal from the probe so as to establish a reference level; c) incubating the probe:HSP90 complex with the test compound; d) measuring the signal from the probe; e) comparing the signal from step d) with the reference level, a modulation of the signal indicating that the test compound binds to HSP90. Also disclosed is a probe which is labeled with a detectable label and/or an affinity tag.

### FIELD OF THE INVENTION

The present invention concerns drug discovery and therapeutic screening assays to identify potential therapeutic agents, particularly those agents that are useful as inhibitors of JNK activation.

### BACKGROUND OF THE INVENTION

Activation of Jun kinase (JNK) has emerged as a central event in neuronal apoptosis (reviewed in (Davis, 2000; Harper and LoGrasso, 2001)). The role of JNK, mechanisms of its activation, and the subsequent events leading to apoptosis has been particularly well elucidated in sympathetic neurons withdrawn from nerve growth factor (NGF). NGF withdrawal results in activation of small GTPases, such as Rac1 (Bazenet et al., 1998), and the subsequent activation of a JNK signaling module that ultimately results in phosphorylation of transcription factors that include c-Jun (Eilers et al., 1998; Eilers et al., 2001; Estus et al., 1994; Ham et al., 1995; Harding et al., 2001). The phosphorylated transcription factors function in part to facilitate production of BH3-domain-only proteins, which are pro-apoptotic members of the Bcl-2 family (Harris and Johnson, 2001; Putcha et al., 2001; Whitfield et al., 2001). Induction of BH3-domain-only proteins leads to the release of mitochondrial contents and thereby initiates the intrinsic apoptotic cascade.

The p75 neurotrophin receptor (p75NTR) and its downstream interacting partner, NRAGE can initiate signaling events leading to neuronal apoptosis (reviewed in (Roux and Barker, 2002)). Several groups have established that in primary neurons and PC12 cells, p75NTR- and NRAGE-induced cell death occurs through induction of the JNK pathway, BH3-domain-only protein activation, and release of mitochondrial contents (Bhakar et al., 2003; Salehi et al., 2000; Salehi et al., 2002).

As JNK plays a central role in neuronal cell death considerable attention has been focused on developing strategies to attenuate JNK signaling, including the development of small molecule inhibitors of JNK activity. Several kinase inhibitors that target elements of the JNK activation pathway have emerged (Bennett et al., 2001; Maroney et al., 2001) and some of these compounds function as anti-apoptotic compounds within in vitro and in vivo models (Saporito et al., 2002; Wang et al., 2004). Another approach is to identify endogenous inhibitors of JNKs that attenuate JNK pathway signaling. Several proteins that directly modulate JNK signaling have been identified (Dickens et al., 1997; Monaco et al., 1999; Muda et al., 1996; Shim et al., 1996; Shim et al., 2000) and the best characterized of these is heat shock protein-70 (HSP70). HSP70 expression is often induced in cells exposed to stressful stimuli, and its role in reducing stress-induced damage through its chaperone function is well established (Young et al., 2004). Independent of its chaperone function, HSP70 can directly bind and inhibit JNK and thereby reduce apoptosis induced by a variety of insults (Gabai et al., 2002; Gabai et al., 2000; Parcellier et al., 2003; Park et al., 2001; Yaglom et al., 1999).

Consequently, there remains a need for assays and screening methods to identify therapeutic agents which inhibit JNK and impact either neuronal apoptosis or dysfunction resulting from physical trauma or disease. To date, small molecule development has been limited to kinase inhibitors of JNK or related upstream kinases. Screens for non-kinase targets have been limited to cellular screens which yield non-molecularly targeted compounds.

### SUMMARY OF THE INVENTION

We herein disclose that Heat Shock Protein-90 (HSP90) represents a novel molecular target for a family of neuroprotective compounds characterized by imidazo[2,1-b-]1,3,4-thiadiazole-2-sulfonamides, imidazo[2,1-b-]1,3,4-thiadiazole-2-sulfones and imidazo[2,1-b-]1,3,4-thiadiazole-2-sulfoxides. The site of binding does not appear to be the N-terminal ATP binding site, as characterized for other HSP90 inhibitors. Furthermore, these finding defines a novel mechanism of neuronal protection mediated by binding to HSP90. We herein disclose several biochemical binding assays useful in the identification of compounds which bind to this novel HSP90 binding site.

We have previously disclosed in WO 03/051,890 A1 and WO 2004/111,060 A1, that imidazo[2,1-b-]1,3,4-thiadiazole-2-sulfonamides, imidazo[2,1-b]-1,3,4-thiadiazole-2-sulfones and imidazo[2,1-b-]1,3,4-thiadiazole-2-sulfoxides, are useful in reducing NGF-withdrawal induced apoptosis of neonatal sympathetic neurons. These compounds were also shown to be useful in the treatment of several neurodegenerative diseases such in the treatment of peripheral neuropathies and optical ischemia. We herein disclose a novel use of the aforesaid compounds as probes or labeled probes in screening methods for JNK activation inhibitors by virtue of their unexpected binding to a novel binding site of HSP90.

Accordingly in one embodiment of the present invention, there is provided an assay for identifying compounds that modulate a neuronal apoptotic pathway, the assay comprising:
a) contacting HSP90 protein with a probe to form a probe:HSP90 complex, the probe being displaceable by a test compound;
b) measuring a signal from the probe so as to establish a reference level;
c) incubating the probe:HSP90 complex with the test compound;
d) measuring the signal from the probe;
e) comparing the signal from step d) with the reference level, a modulation of the signal indicating that the test compound binds to the HSP90 protein,
   wherein the probe is a compound of Formula 1, as described below,
   and wherein the probe of Formula 1 is labeled with a detectable label and/or an affinity tag.

According to one aspect of the present invention, there is provided an assay for identifying compounds that inhibit the apoptotic JNK signaling pathway, the assay comprising:
a) contacting HSP90 protein with a probe to form a probe:HSP90 complex, the probe being displaceable by a test compound;
b) measuring a signal from the probe so as to establish a reference level;
c) incubating the probe:HSP90 complex with the test compound;
d) measuring the signal from the probe;
e) comparing the signal from step d) with the reference level, a modulation of the signal indicating that the test compound binds to the HSP90 protein,
   wherein the probe is a compound of Formula 1, as described below,
   and wherein the probe of Formula 1 is labeled with a detectable label and/or an affinity tag.

According to another embodiment of the present invention there is provided an assay for identifying compounds that modulate a neuronal apoptotic pathway, the assay comprising:
a) providing a cell expressing HSP90 protein;
b) contacting the cell with a test compound;
c) determining whether HSP70 protein is expressed in the cell, the expression of the HSP70 protein being an indication that the test compound binds to the HSP90 protein.

According to one aspect of the present invention there is provided an assay for identifying compounds that modulate a neuronal apoptotic pathway, the assay comprising:
a) providing a cell expressing HSP90 protein;
b) contacting the cell with a test compound;
c) determining whether HSP70 protein is expressed in the cell, the expression of the HSP70 protein being an indication that the test compound binds to the HSP90 protein.

According to another aspect of the present invention there is provided a method of modulating a neuronal apoptotic pathway, the method comprising: increasing the expression of HSP70 protein by binding a compound, according to Formula 1a, to HSP90 protein, so as to cause modulation of the neuronal apoptotic pathway.

According to another embodiment of the present invention, there is provided a probe, according to Formula I:

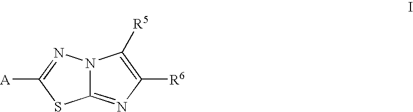

or a salt thereof,
wherein:
A is
   1) —S(O)₂NR¹R²; or
   2) —S(O)ₙR³;
n is 1 or 2;
m is 1 to 20;
Y is NH, O or S;
R¹ and R² are independently selected from:
   1) H, or
   2) C₁-C₆ alkyl;
R³ is:
   1) C₁-C₆ alkyl,
   2) haloalkyl,
   3) aryl, or
   4) heteroaryl,
      wherein the alkyl is optionally substituted with one or more R¹⁵ substituents; and the aryl and heteroaryl are optionally substituted with one or more R²⁰ substituents;
R⁵ is:
   1) H,
   2) halogen,
   3) C₁-C₆ alkyl, or
   4) aryl;
R⁶ is
   1) H,
   2) C₁-C₆ alkyl
   3) C₁-C₆ haloalkyl,
   4) adamantyl,
   5) aryl, or
   6) heteroaryl,
      wherein the aryl and the heteroaryl are optionally substituted with one or more substituents independently selected from R²⁰;
R⁷ is
   1) H,
   2) haloalkyl,
   3) C₁-C₆ alkyl,
   4) aryl,
   5) heteroaryl or
   6) heterocyclyl,
      wherein the alkyl is optionally substituted with one or more R¹⁵ substituents; and wherein the aryl, heteroaryl and heterocyclyl is optionally substituted with one or more R^{2c} substituents;
R¹⁰ is
   1) C₁-C₆ alkyl,
   2) C₃-C₇ cycloalkyl,
   3) haloalkyl,
   4) C₂-C₆ alkenyl,
   5) C₂-C₆ alkynyl,
   6) C₅-C₇ cycloalkenyl,
   7) aryl,
   8) heteroaryl, or
   9) heterocyclyl,
      wherein the alkyl, cycloalkyl, alkenyl, alkynyl, cycloalkenyl are optionally substituted with one or more R¹⁵ substituents, and the aryl, heteroaryl, and heterocyclyl are optionally substituted with one or more R²⁰ substituents;
R¹¹ and R¹² are independently selected from:
   1) H,
   2) C₁-C₆ alkyl,
   3) C₃-C₇ cycloalkyl,
   4) haloalkyl,
   5) aryl,
   6) heteroaryl,
   7) heterocyclyl,
   8) C(O)—C₁-C₆ alkyl
   9) C(O)—C₃-C₇ cycloalkyl
   10) C(O)-aryl,
   11) C(O)-heteroaryl,
   12) C(O)-heterocyclyl,
   13) C(O)Y—C₁-C₆ alkyl
   14) C(O)Y—C₃-C₇ cycloalkyl
   15) C(O)Y-aryl,
   16) C(O)Y-heteroaryl,
   17) C(O)Y-heterocyclyl,
      wherein the alkyl and the cycloalkyl are optionally substituted with one or more R¹⁵ substituents, and the aryl, heteroaryl, and heterocyclyl are optionally substituted with one or more R²⁰ substituents;
      or R¹¹ and R¹² together with the nitrogen atom to which they are bonded form a five, six or seven membered heterocyclic ring optionally substituted with one or more R²⁰ substituents;
R¹⁴ is
   1) H, or
   2) C₁-C₆ alkyl;
R¹⁵ is
   1) NO₂,
   2) CN,
   3) halogen,
   4) C₃-C₇ cycloalkyl,
   5) haloalkyl,
   6) aryl,
   7) heteroaryl,
   8) heterocyclyl,
   9) OR⁷,
   10) S(O)ₙR¹⁰,
   11) NR¹¹R¹²,
   12) CHO,
   13) C(O)R¹⁰,
   14) CO₂R¹⁴,
   15) CONR¹¹R¹², or
   16) S(O)ₙNR¹¹R¹²,
      wherein the aryl and heteroaryl are optionally substituted with one or more R²⁵ substituents;
R²⁰ is
   1) NO₂,
   2) CN,
   3) N₃,
   4) B(OR¹⁴)₂,
   5) adamantyl,
   6) halogen,
   7) C₁-C₆ alkyl,
   8) C₂-C₆ alkenyl,
   9) C₂-C₄ alkynyl,
   10) C₃-C₇ cycloalkyl,
   11) C₃-C₇ cycloalkenyl,
   12) aryl,
   13) heteroaryl,
   14) heterocyclyl,
   15) haloalkyl,
   16) OR⁷,
   17) SR⁷,
   18) S(O)ₙR¹⁰,
   19) NR¹¹R¹²
   20) CHO,
   21) C(O)R¹⁰,
   22) C(O)OR¹⁴,
   23) CONR¹¹R¹²,
   24) S(O)ₙNR¹¹R¹²,
   25) NR¹⁴C(O)R¹⁰,
   26) NR¹⁴S(O)₂R¹⁰,
   27) OC(O)R¹⁰,
   28) SC(O)R¹⁰,
   29) —O—(C₁-C₆alkyl-O)ₘR⁴, or
   30) —O—(C₁-C₆ alkyl-O)ₘ—C₁-C₆ alkyl-OR¹⁴,
      wherein the alkyl and the cycloalkyl are optionally substituted with one or more R¹⁵ substituents; and wherein the aryl, heteroaryl and the heterocyclyl are optionally substituted with one or more R²⁵ substituents;
R²⁵ is
   1) halogen,
   2) NO₂,
   3) CN,
   4) B(OR¹⁴)₂,
   5) haloalkyl,
   6) C₁-C₆ alkyl,
   7) C₂-C₆ alkenyl,
   8) C₂-C₄ alkynyl,
   9) OR⁷,
   10) SR⁷
   11) NR¹¹R¹²,
   12) CHO,
   13) C(O)R¹⁰,
   14) C(O)OR¹⁴,
   15) CONR¹¹R¹²,
   16) S(O)₂R¹⁰,
   17) S(O)₂NR¹¹R¹²,
   18) NR¹⁴COR¹⁰,
   19) NR¹⁴S(O)₂R¹⁰,
   20) OC(O)R¹⁰, or
   21) SC(O)R¹⁰;
      wherein the probe may be labeled with a detectable label or an affinity tag and wherein the probe binds to HSP90 protein.

Typically, the assays, as described above, in which the signal may be chosen from: fluorescence, resonance energy transfer, time resolved fluorescence, radioactivity, fluorescence polarization, plasma resonance, chemiluminescence, nuclear magnetic resonance (NMR) spectroscopy, mass spectroscopy (MS) and the like.

### BRIEF DESCRIPTION OF THE DRAWINGS

Further aspects and advantages of the present invention will become better understood with reference to the description in association with the following Figures, wherein:

FIG. 1 is a graph showing that compound 1 inhibits NGF-withdrawal induced cell death. Cultures of rat SCG neurons (see Materials and Methods) initially maintained in 10 ng/ml of NGF were withdrawn from NGF and maintained in media containing compound 1 at the concentrations indicated for 48 hours. Cellular viability was assayed using an MTS assay. (*=P<0.01 relative to no compound 1). Experiments were performed three times with identical results.

FIGS. 2A and 2B are graphs showing that compound 1 inhibits p75NTR or NRAGE induced JNK activation and cell death. Compound 1 inhibits NRAGE (A) or p75NTR (B) mediated cell death. PC12^{rtTA} cells were infected with recombinant adenoviruses expressing full-length NRAGE (AdNRG) at 5 MOI, full length p75NTR (Adp75) at 50 or 100 MOI, or the control protein β-galactosidase (AdLacZ) (at 5 MOI in (A) and 50 MOI in (B)). At time of infection, the cells were treated with an increasing concentration of compound 1, or with vehicle alone, as indicated. After 40 hours, cells were assayed for death using the lactate dehydrogenase (LDH) assay; untreated cells (Un) and cells treated with 1% Triton X-100 (Tx) were used to delineate the output range of the assay. Results are normalized relative to those obtained with 1% Triton (Tx), and represent the mean±standard deviation of a representative experiment performed in triplicate (*=P<0.005 relative to no compound 1).

FIGS. 2C and 2D are photographs of Western blots which illustrate that compound 1 inhibits NRAGE- and p75NTR-induced JNK activation. C, D: Compound 1 inhibits NRAGE- and p75NTR-induced JNK activation. PC12^{rtTA} cells were infected with AdNRG at 5 MOI (C), with Adp75 at 50 MOI (D), or with AdLacZ at an equivalent MOI for each experiment, as indicated. At time of infection, the cells were treated with increasing concentrations of compound 1, or with vehicle alone, as indicated. Thirty hours after infection, cells were lysed, normalized for protein content, and analyzed for levels of phospho-Jun, total c-Jun, cleaved caspase-3, JNK, IkBα, NRAGE (C) and p75NTR (D). by immunoblotting. Experiments in Panel A-D were performed three times with essentially identical results.

FIG. 3 is a graph showing that compound 1 inhibits JNK-dependent cell death induced by chemotherapeutic drugs. Cells were infected with AdNRG at 5 MOI or treated with paclitaxel, cisplatin, or doxorubicin (Dox) at the indicated μM concentrations. At the time of treatment, compound 1 or vehicle was added to the cultures, as indicated. After 40 hours, cells were assayed for death using the LDH assay. Results are normalized relative to those obtained with 1% Triton (Tx), and represent the mean±standard deviation of a representative experiment performed in triplicate (*=P<0.01 relative to 0 μM compound 1 for each insult). Experiments were performed twice with identical results.

FIGS. 4A-4C are photographs of Western blots which illustrate that compound 1 treatment of PC12 cells induces expression of HSP70. A: PC12 cells were infected with 5 MOI of AdNRG (NRG) or AdLacZ (LacZ) as indicated. Cells were left untreated (0) or treated with 40 μM compound 1 at time of infection (30), one hour before lysis (1), or at both times (30+1). Cells were lysed thirty hours after infection, normalized for protein content, and analyzed for levels of phospho-Ser⁶³ c-Jun and total c-Jun, total JNK, IkBα, and NRAGE by immunoblotting. Lysates from untreated cells are indicated by ‘−’. B: PC12 cells were treated with the indicated concentrations of compound 1 for 18 hours and then lysed, normalized for protein content, and analyzed for levels of HSP70 by immunoblotting. Lysates from untreated cells are indicated by ‘−’. C: RNA isolated from PC12^{rtTA} cells treated with increasing concentrations of compound 1 for 18 hours was analyzed by rtPCR using primers specific to HSP70, HSP25, HSP90 or actin, as indicated. Experiments in Panel A and B were performed three times and that in Panel C was performed twice, with essentially identical results. D: PC12^{rtTA} cells were infected with AdNRG at 5 MOI, Adp75 at 50 MOI, or the control virus AdLacZ at 50 MOI. At time of infection, the cells were treated with increasing concentrations of compound 1, or with vehicle alone, as indicated. Thirty hours after infection, cells were lysed, normalized for protein content, and analyzed for levels of c-Jun phosphorylation, HSP70, HSP25, HSP40, NRAGE, p75NTR, JNK-1 and IkBα, as indicated.

FIG. 5A is a graph showing that compound 1 binds HSP90 and causes HSF-1 dependent expression of HSP70.

FIGS. 5B and 5C are photographs of Western blots showing that compound 1 binds HSP90 and causes HSF1-dependent expression of HSP70 and HSP25. A: Wild-type or HSF1 null MEFs were transfected with pGL3B-HSP70, an HSP70 promoter reporter construct, or parental vector (pGL3B), then treated with 40 μM of compound 1 for 24 hours and analyzed for luciferase content as described in Materials and Methods (*=P<0.005 relative to 0 compound 1; **=P<0.005 relative to wild-type MEFs treated with 40 uM compound 1. B: Wild-type or HSF1 null MEFs were treated with increasing concentrations of compound 1 for 24 hours, cells were lysed, normalized for protein content, and analyzed for levels of HSP70, HSP40, and HSP25 as indicated. C: Compound 6-conjugated Sepharose beads were incubated with purified HSP90 for 2 hours, washed, and levels of bound HSP90 were analyzed by immunoblot. Con=Control beads, Alone=Compound 6-beads alone, Comp=Compound 1 beads+competing compound 1 (at 80 μM). Experiments in Panel A-C were performed three times with identical results.

FIGS. 6A, 6B and 6C are graphs and photographs of Western blots which illustrate that compound 1 analogues inhibit JNK activation and induce expression of HSP70. A: PC12^{rtTA} cells were infected with 5 MOI of AdNRG, 50 MOI of Adp75 or 50 MOI of the control virus AdLacZ. At time of infection, the cells were treated with an increasing concentrations of compound 1, or its analogues, compound 2, compound 3, compound 4, or with vehicle alone, as indicated. Thirty hours after infection, cells were lysed, normalized for protein levels, and analyzed for levels of HSP70, HSP25 and JNK-1, as indicated. B: PC12^{rtTA} cells were treated as above and after 40 hours infection were assayed for death using the lactate dehydrogenase (LDH) assay. Results are normalized relative to those obtained with 1% Triton, and represent the mean±standard deviation of a representative experiment performed in triplicate (*=P<0.025 relative to ‘0’ for each compound concentration). C: PC12^{rtTA} cells were infected with 50 MOI of LacZ or 5 MOI of AdNRG50 and at the time of infection, were treated with 40 μM compound 1, or its analogues, compound 2, compound 3, compound 4, or with vehicle alone, as indicated. Thirty hours after infection, cells were lysed, normalized for protein levels, and were analyzed for phosphorylated c-Jun, total c-Jun, NRAGE and JNK-1, as indicated. Experiments in Panel A and C were performed twice and that in B were performed twice, with essentially identical results.

FIGS. 7A, 7B and 7C are photographs of Western blots which illustrate that compound 1 analogues inhibit JNK activation and induce expression of HSP70. A: PC12^{rtTA} cells were transfected with RNAi directed against HSP70 or with control RNA and then 24 hours later were infected with 5 MOI of AdNRG or 50 MOI of the control virus AdLacZ. At time of infection, the cells were treated with 40 μM compound 2 or with a vehicle control, as indicated. After 30 hours, cells were lysed, normalized for protein levels, and analyzed for phosphorylated Jun, HSP70, HSP25 NRAGE and actin levels. B: PC12^{rtTA} cells were transfected with an expression construct driving GST-Jun. After 24 hours, cells were infected with 5 MOI of AdNRG or 50 MOI of the control virus AdLacZ or were left uninfected. After a further 24 hours, uninfected cells were left untreated or were exposed to 10 ng/ml TNF or 300 mM sorbitol for 15 minutes. Cells were then lysed, normalized for protein levels, and GST-jun was recovered by glutathione pullout and analyzed for phosphorylation using a antibody directed against c-Jun pSer63 by immunoblotting. Equivalent pullout of GST-Jun was confirmed by immunoblotting with an antibody directed against c-Jun and GST. C: PC12^{rtTA} cells were co-transfected with an expression construct driving GST-Jun and with RNAi directed against HSP70 or with control RNA, as indicated. After 24 hours, cells were infected with 5 MOI of AdNRG or 50 MOI of the control virus AdLacZ. At time of infection, the cells were treated with 40 μM compound 2 or with a vehicle control, as indicated. After 30 hours, cells were lysed, normalized for protein levels, and GST-jun was recovered by glutathione pullout and analyzed for phosphorylation using an antibody directed against c-Jun pSer63 by immunoblotting. Equivalent pullout of GST-Jun was confirmed by immunoblotting with an antibody directed against total Jun. Lysates levels of HSP70, HSP25, HSP40, NRAGE and actin was determined by immunoblot. Experiments in A and C were performed three times and experiment in B was performed twice, all with essentially identical results.

FIGS. 8A and 8B are photographs of Western blots which illustrate that compound 1 and Geldanamycin differ in their mechanism of action. A: Purified HSP90 protein was pre-incubated with ATP, geldanamycin (1 μM) or with the indicated concentrations of compound 1 (indicated in μM) then mixed with γATP-Sepharose beads. Bound HSP90 was eluted HSP90 content was determined by immunoblotting. B: PC12 cells were exposed to increasing concentrations of compound 1 and geldanamycin (indicated, in μM) for 16 hours, lysed and analyzed by immunoblotting for levels of phosphorylated and total Akt, HSP70, HSP90 and Actin, as indicated.

FIG. 9 is a STD 1H NMR Spectrum of Compound 1 and 20 with HSP90 protein. STD NMR experiments were performed at 19° C. on a Varian INNOVA 500 MHz spectrometer equipped with a triple resonance HCN cold probe. A 1D saturation transfer difference pulse sequence with internal subtraction via phase cycling was employed. Residual HDO signal was removed using a W5 WATERGATE pulse sequence, with a 150 ms interpulse delay. On resonance irradiation of the protein was performed at −0.5 ppm, with off-resonance irradiation at 34 ppm. The irradiation power was (g/2p)B₁=93 Hz applied through a train of 50 ms eburp1 pulses with a 1 ms delay between the pulses. The total presaturation time was 5.1 s. The relaxation delay was set to 0.1 s. A 30 ms spin-lock pulse with a strength of (g/2p)B₁=4600 Hz was used to eliminate background protein signals. The total number of scans was between 1536, with a sweep width of 16 ppm. Spectra were multiplied by a 1 Hz exponential line-broadening function prior to Fourier transform. Compounds 1 (85 uM) and 20 (85 uM) and HSP90 protein (0.85 uM) in 20 mM sodium phosphate buffer (pH 6.7) was used. A) STD 1H NMR spectrum, B) preset reference, and C) STD reference-no saturation/difference.

### DETAILED DESCRIPTION OF THE INVENTION

### Definitions

Unless otherwise specified, the following definitions apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus the phrase “consisting of” indicates that the listed elements are required or mandatory and that no other elements may be present.

As used herein, the term “alkyl” is intended to include both branched and straight chain saturated aliphatic hydrocarbon groups having the specified number of carbon atoms, for example, C₁-C₁₅ as in C₁-C₁₅-alkyl is defined as including groups having 1, 2, 3, 4, 5, 6, 7, 8, 9, 10, 11, 12, 13, 14, or 15 carbons in a linear or branched arrangement, and C₁-C₆ as in C₁-C₆-alkyl is defined as including groups having 1, 2, 3, 4, 5 or 6 carbons in a linear or branched arrangement, and C₁-C₄ as in C₁-C₄ alkyl is defined as including groups having 1, 2, 3, or 4 carbons in a linear or branched arrangement, and C₁-C₃ as in C₁-C₃ alkyl is defined as including groups having 1, 2, or 3 carbons in a linear or branched arrangement. Examples of alkyl as defined above include, but are not limited to, methyl, ethyl, n-propyl, i-propyl, n-butyl, t-butyl, i-butyl, pentyl and hexyl.

As used herein, the term, “alkenyl” is intended to mean unsaturated straight or branched chain hydrocarbon groups having the specified number of carbon atoms therein, and in which at least two of the carbon atoms are bonded to each other by a double bond, and having either E or Z regeochemistry and combinations thereof. For example, C₂-C₆ as in C₂-C₆ alkenyl is defined as including groups having 1, 2, 3, 4, 5, or 6 carbons in a linear or branched arrangement, at least two of the carbon atoms being bonded together by a double bond. Examples of C₂-C₆ alkenyl include ethenyl (vinyl), 1-propenyl, 2-propenyl, 1-butenyl and the like.

As used herein, the term “alkynyl” is intended to mean unsaturated, straight chain hydrocarbon groups having the specified number of carbon atoms therein and in which at least two carbon atoms are bonded together by a triple bond. For example C₂-C₄ as in C₂-C₄ alkynyl is defined as including groups having 2, 3, or 4 carbon atoms in a chain, at least two of the carbon atoms being bonded together by a triple bond. Examples of such alkynyls include ethynyl, 1-propynyl, 2-propynyl and the like.

As used herein, the term “cycloalkyl” is intended to mean a monocyclic saturated aliphatic hydrocarbon group having the specified number of carbon atoms therein, for example, C₃-C₇ as in C₃-C₇cycloalkyl is defined as including groups having 3,4,5,6, or 7 carbons in a monocyclic arrangement. Examples of C₃-C₇ cycloalkyl as defined above include, but are not limited to, cyclopropyl, cyclobutyl, cyclopentyl, cyclohexyl and cycloheptyl.

As used herein, the term “cycloalkenyl” is intended to mean a monocyclic saturated aliphatic hydrocarbon group having the specified number of carbon atoms therein, for example, C₃-C₇ as in C₃-C₇cycloalkenyl is defined as including groups having 3,4,5,6, or 7 carbons in a monocyclic arrangement. Examples of C₃-C₇ cycloalkenyl as defined above include, but are not limited to, cyclopentenyl, and cyclohexenyl.

As used herein, the term “halo” or “halogen” is intended to mean fluorine, chlorine, bromine and iodine.

As used herein, the term “haloalkyl” is intended to mean an alkyl as defined above, in which each hydrogen atom may be successively replaced by a halogen atom. Examples of haloalkyls include, but are not limited to, CH₂F, CHF₂ and CF₃.

As used herein, the term “aryl”, either alone or in combination with another radical, means a carbocyclic aromatic monocyclic group containing 6 carbon atoms which may be further fused to a second 5- or 6-membered carbocyclic group which may be aromatic, saturated or unsaturated. Aryl includes, but is not limited to, phenyl, indanyl, 1-naphthyl, 2-naphthyl and tetrahydronaphthyl. The fused aryls may be connected to another group either at a suitable position on the cycloalkyl ring or the aromatic ring. For example:

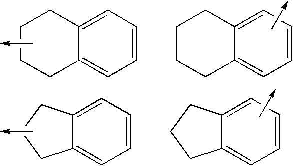

Arrowed lines drawn from the ring system indicate that the bond may be attached to any of the suitable ring atoms. Also included in the definition of aryl are biphenyls in which two phenyl groups bonded together at any one of the available sites on the phenyl ring. For example:

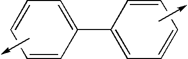

As used herein, the term “heteroaryl” is intended to mean a monocyclic or bicyclic ring system of up to ten atoms, wherein at least one ring is aromatic, and contains from 1 to 4 hetero atoms selected from the group consisting of O, N, and S. The heteroaryl substituent may be attached either via a ring carbon atom or one of the heteroatoms. Examples of heteroaryl groups include, but are not limited to thienyl, benzimidazolyl, benzo[b]thienyl, furyl, benzofuranyl, pyranyl, isobenzofuranyl, chromenyl, xanthenyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, napthyridinyl, quinoxalinyl, quinazolinyl, cinnolinyl, pteridinyl, isothiazolyl, isochromanyl, chromanyl, isoxazolyl, furazanyl, indolinyl, isoindolinyl, thiazolo[4,5-b]-pyridine, benzo[d][1,3]dioxole, 2,3-dihydrobenzo[b][1,4]dioxine, 3,4-dihydro-2H-benzo[b][1,4]dioxepine, or coumarinyl.

Alkyl substitutents may include fluoroscein derivatives including, but not limited to:

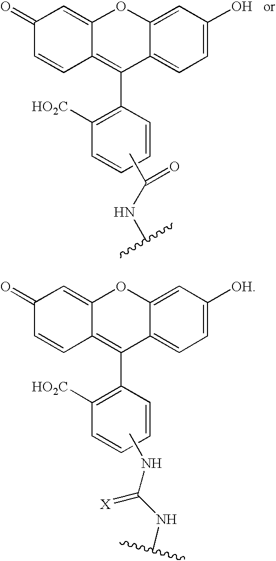

Also included in this definition are BODIPY or other fluorescent derivatives such as, but not limited to:

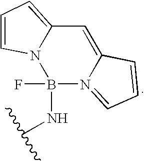

As used herein, the term “heterocycle”, “heterocyclic” or “heterocyclyl” is intended to mean a 5, 6, or 7 membered non-aromatic ring system containing from 1 to 4 heteroatoms selected from the group consisting of O, N and S. Examples of heterocycles include, but are not limited to pyrrolidinyl, tetrahydrofuranyl, piperidyl, pyrrolinyl, piperazinyl,

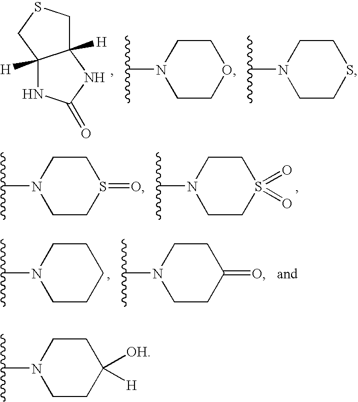

imidazolidinyl, morpholinyl, imidazolinyl, pyrazolidinyl, pyrazolinyl,

As used herein, the term “heterobicycle” either alone or in combination with another radical, is intended to mean a heterocycle as defined above fused to another cycle, be it a heterocycle, an aryl or any other cycle defined herein. Examples of such heterobicycles include, but are not limited to, coumarin, benzo[d][1,3]dioxole, 2,3-dihydrobenzo[b][1,4]dioxine and 3,4-dihydro-2H-benzo[b][1,4]dioepine.

As used herein, the term “heteroatom” is intended to mean O, S or N.

As used herein, the term “probe” is intended to mean a compound of Formula I which is labeled with either a detectable label or an affinity tag, and which is capable of binding, either covalently or non-covalently, to HSP90 protein. When, for example, the probe is non-covalently bound, it may be displaced by a test compound. When, for example, the probe is bound covalently, it may be used to form cross-linked adducts, which may be quantified and inhibited by a test compound.

As used herein, the term “optionally substituted with one or more substituents” or its equivalent term “optionally substituted with at least one substituent” is intended to mean that the subsequently described event of circumstances may or may not occur, and that the description includes instances where the event or circumstance occurs and instances in which it does not. The definition is intended to mean from zero to five substituents.

If the substituents themselves are incompatible with the synthetic methods of the present invention, the substituent may be protected with a suitable protecting group (PG) that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3^{rd} ed.), John Wiley & Sons, NY (1999), which is incorporated herein by reference in its entirety. Examples of protecting groups used throughout include, but are not limited to Fmoc, Bn, Boc, CBz and COCF₃. In some instances, a substituent may be specifically selected to be reactive under the reaction conditions used in the methods of this invention. Under these circumstances, the reaction conditions convert the selected substituent into another substituent that is either useful in an intermediate compound in the methods of this invention or is a desired substituent in a target compound.

As used herein, the term “subject” is intended to mean humans and non-human mammals such as primates, cats, dogs, swine, cattle, sheep, goats, horses, rabbits, rats, mice and the like.

As used herein, the term “pharmaceutically acceptable carrier, diluent or excipient” is intended to mean, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, or encapsulating agent, such as a liposome, cyclodextrins, encapsulating polymeric delivery systems or polyethyleneglycol matrix, which is acceptable for use in the subject, preferably humans.

As used herein, the term “pharmaceutically acceptable salt” is intended to mean both acid and base addition salts.

As used herein, the term “pharmaceutically acceptable acid addition salt” is intended to mean those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

As used herein, the term “pharmaceutically acceptable base addition salt” is intended to mean those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

As used herein, the term “HSP90 protein” is intended to mean 90 kilo Dalton heat shock proteins, which are ubiquitous chaperone proteins, which bind and hydrolyze ATP. The Hsp90 family of proteins includes four known members: Hsp90-alpha and -beta, Grp94, and Trap-1. Hsp90s are understood to regulate client proteins involved in cellular signaling (Wegele et al., 2004, Rev Physiol Biochem Pharmocol 151:1-44). These client proteins include key proteins involved in signal transduction, cell cycle control, and transcriptional regulation (Burrows et al., 2004, Cell Cycle, 3:e20-e26; Pratt et al., 2004, Cellular Signalling, 16:857-872). Also included in the term “HSP90 protein” are substantially pure preparations of HSP90, fragments thereof, and recombinant HSP90 proteins including full and partial length HSP90 fused to GST, 6-Histidine and other tags, prepared by method known in the art.

As used herein the term “HSP70 protein” is intended to mean a heat shock protein 70 kilo Dalton heat shock protein 70 which, like the HSP90 proteins, also functions as a molecular chaperone by interacting with the cellular proteins in an ATP-dependent manner.

As used herein, the term “HSP90 binding” is intended to mean the action of a compound or a probe of the present invention upon HSP90. The effects of a compound binding to HSP90 may include altered stability of client proteins and effects on co-chaperone proteins such as Heat Shock Factor 1 (HSF1) leading to increased expression of HSP70.

As used herein, the term “neuronal apoptosis pathway” is intended to mean a pathway that regulates the apoptosis of neuronal cells. An example of such a neuronal apoptosis pathway includes, but is not limited to, the apoptotic JNK signaling pathway.

As used herein, the term “apoptosis” or “programmed cell death” is intended to mean the regulated process of cell death wherein a dying cell displays a set of well-characterized biochemical hallmarks that include cell membrane blebbing, cell soma shrinkage, chromatin condensation, and DNA laddering, as well as any caspase-mediated cell death.

As used herein, the term “modulate” or “modulating” is intended to mean the treatment, prevention, inhibition, suppression, enhancement or induction of a function or condition using the compounds or probes of the present invention. For example, the compounds or probes of the present invention can bind to HSP90 protein and increase the expression of HSP70 protein from one level to another level and causing inhibition of the neuronal apoptosis pathway, thereby modulating apoptosis of neuronal cells.

As used herein, the term “modulating apoptosis” is intended to mean increasing or decreasing the number of cells that apoptose in a given cell population either in vitro or in vivo. Examples of cell populations include, but are not limited to, neuronal cells and the like. It will be appreciated that the degree of apoptosis modulation provided by an apoptosis-modulating compound of the present invention in a given assay will vary, but that one skilled in the art can determine the statistically significant change in the level of apoptosis that identifies a compound that modulates apoptosis otherwise limited by other regulators of the neuronal apoptosis pathway.

The compounds or probes of the present invention, or their pharmaceutically acceptable salts may contain one or more asymmetric centers, chiral axes and chiral planes and may thus give rise to enantiomers, diastereomers, and other stereoisomeric forms and may be defined in terms of absolute stereochemistry, such as (R)- or (S)- or, as (D)- or (L)- for amino acids. The present invention is intended to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. The racemic mixtures may be prepared and thereafter separated into individual optical isomers or these optical isomers may be prepared by chiral synthesis. The enantiomers may be resolved by methods known to those skilled in the art, for example by formation of diastereoisomeric salts which may then be separated by crystallization, gas-liquid or liquid chromatography, selective reaction of one enantiomer with an enantiomer specific reagent. It will also be appreciated by those skilled in the art that where the desired enantiomer is converted into another chemical entity by a separation technique, an additional step is then required to form the desired enantiomeric form. Alternatively specific enantiomers may be synthesized by asymmetric synthesis using optically active reagents, substrates, catalysts, or solvents or by converting one enantiomer to another by asymmetric transformation.

Certain compounds or probes of the present invention may exist in Zwitterionic form and the present invention includes Zwitterionic forms of these compounds and mixtures thereof

### I. Screening Assays

The mechanism of action of compounds of as described in WO 03/051,890 A1 and WO 2004/111,060 A1, and their pharmaceutically acceptable salts, has now been elucidated based on a novel use of probes of Formula I, which are described in more detail below. In one example, we discovered unexpectedly that the probes of the present invention bind to non-ATPase binding site of the HSP90 protein. One compound of Formula I completely blocked apoptosis induced by the p75 neurotrophin receptor (p75NTR) or its cytosolic interactor, NRAGE and our mechanistic studies revealed that treatment with the compound strongly attenuated JNK and caspase-3 activation. Specifically, compounds of this class induces production of heat shock protein 70 (HSP70), an endogenous inhibitor of JNK. We have shown that compound 1 induces HSP70 by binding HSP90 and thereby induces HSF1-dependent expression of HSP70 mRNA. We have established that accumulation of HSP70 is required for the compound 1-induced blockade of JNK signaling. We show that compound 1 facilitates HSP70 production while retaining HSP90 chaperone activity. Further, compounds 1 and 10 to 17 inhibit staurosposine induced induction of JNK in PC12 cells. Further, we demonstrate that compounds 1, 2, 5, 12, 13, 15, and 20 directly bind to HSP90 protein as determined by STD ¹H NMR spectroscopy. These studies establish that select imidazo[2,1-b-]1,3,4-thiadiazole-2-sulfonamides, imidazo[2,1-b-]1,3,4-thiadiazole-2-sulfones and imidazo[2,1-b-]1,3,4-thiadiazole-2-sulfoxides by binding HSP90 and inducing the expression of HSP70 which directly inhibits JNK activation and apoptosis by a unique mechanism involving induced expression of HSP70 proteins. The probes of the present invention can be displaced by test compounds that also inhibit the JNK signaling pathway that is critical in apoptosis regulation, and thus be useful in screening assays for therapeutic compounds which inhibit JNK activation.

In one embodiment of the present invention, there is provided an assay for identifying compounds that modulate a neuronal apoptotic pathway, the assay comprising:
a) contacting an HSP90 protein and a probe to form a probe:HSP90 complex, the probe being displaceable by a test compound;
b) measuring a signal from the probe so as to establish a reference level;
c) incubating the probe:HSP90 complex with the test compound;
d) measuring the signal from the probe;
e) comparing the signal from step d) with the reference level, a modulation of the signal indicating that the test compound binds to HSP90,
   wherein the probe is a compound of Formula 1, as described below, and wherein the probe of Formula 1 is labeled with a detectable label and/or an affinity tag.

Competitive screening assays may be done by combining an HSP90 protein and a test compound in a first sample. A second sample comprises a known inhibitor, such as those compounds of Formula Ia, an HSP90 protein and a test compound. The binding of the test compound is determined for both samples, and a change, or difference in binding between the two samples indicates the presence of a test compound which is capable of binding to HSP90 and potentially modulating its activity and/or the expression of HSP70. That is, if the binding of the test compound is different in the second sample relative to the first sample, the test compound is capable of binding to HSP90 protein.

In one example, the binding of the test compound is determined through the use of competitive binding assays. Typically, the competitor is a binding moiety known to bind to HSP90 protein, such as an antibody, peptide, binding partner, ligand, and the like. Under certain circumstances, there may be competitive binding as between the test compound and the binding moiety, with the binding moiety displacing the test compound.

In one example, the test compound may be labeled. Either the test compound, or the competitor, or both, is added first to an HSP90 protein for a time sufficient to allow binding, if present. Incubations may be performed at any temperature which facilitates optimal activity, typically between about 4 and about 40° C. Incubation periods are selected for optimum activity, but may also be optimized to facilitate rapid high throughput screening. Excess reagents are generally removed or washed away. The second component is then added, and the presence or absence of the labeled component is followed, to indicate binding.

In another example, the competitor is added first, followed by the test compound. Displacement of the competitor is an indication the test compound is binding to an HSP90 proteinand thus is capable of binding to, and potentially modulating, the activity of HSP90 protein and causing HSP70 expression. In another example, either component can be labeled. Thus, for example, if the competitor is labeled, the presence of label in the wash solution indicates displacement by the test compound, or if the test compound is labeled, the presence of the label on the support indicates displacement.

In another example, the test compound is added first, with incubation and washing, followed by the competitor. The absence of binding by the competitor may indicate the test compound is bound to as HSP90 protein with a higher affinity. Thus, if the test compound is labeled, the presence of the label on the support, coupled with a lack of competitor binding, may indicate the test compound is capable of binding to HSP90.

It may be valuable to identify a binding site of HSP90 protein. This may be done in a number of ways. In one example, the HSP90 may be fragmented or modified and the assays repeated to identify the necessary components for binding. Alternatively, selected regions of HSP90 protein may be cloned and expressed as individual HSP90 protein fragments. HSP90 is generally referred to as having 3 distinct regions referred to as the N-terminal ATP-binding region, the middle domain, and the C-terminal ATP-binding domain.

Modulation is tested by screening for test compounds that are capable of modulating the activity of HSP90 and/or HSP70 comprising the steps of combining a test compound with HSP90 protein, as above, and determining an alteration in the biological activity of HSP90 and/or HSP70. Thus, in this example, the test compounds should both bind HSP90 protein, and alter its biological or biochemical activity as defined herein. The methods include both in vitro screening methods and in vivo screening of cells for alterations in HSP70 expression or modulation or apoptotic JNK signaling pathway.

As an alternative example, differential screening may be used to identify drug candidates that bind to the native HSP90 protein, but cannot bind to modified HSP90 protein.

Positive controls and negative controls may be used in the assays. Typically, all control and test samples are performed in at least triplicate to obtain statistically significant results. Incubation of all samples is for a time sufficient for the binding of the test compound to the HSP90 protein. Following incubation, all samples are washed free of non-specifically bound material and the amount of bound, generally labeled compound determined. For example, where a radiolabel is employed, the samples may be counted in a scintillation counter to determine the amount of bound compound.

A variety of other reagents may be included in the screening assays. These include reagents like salts, neutral proteins, for example, albumin, detergents, and the like which may be used to facilitate optimal protein-protein binding and/or reduce non-specific or background interactions. Also reagents that otherwise improve the efficiency of the assay, such as protease inhibitors, nuclease inhibitors, anti-microbial agents, and the like, may be used. The mixture of components may be added in any order that provides for the requisite binding.

According to one aspect of the present invention, there is provided an assay for identifying compounds that modulate the apoptotic JNK signaling pathway, the assay comprising:
a) contacting an HSP90 protein with a probe to form a probe:HSP90 complex, the probe being displaceable by a test compound;
b) measuring a signal from the probe so as to establish a reference level;
c) incubating the probe:HSP90 complex with the test compound;
d) measuring the signal from the probe;
e) comparing the signal from step d) with the reference level, a modulation of the signal indicating that the test compound binds to HSP90,
   wherein the probe is a compound of Formula 1, as described below, and wherein the probe of Formula 1 is labeled with a detectable label and/or an affinity tag.

According to another embodiment of the present invention there is provided an assay for identifying compounds that modulate a neuronal apoptotic pathway, the assay comprising:
a) providing a cell expressing HSP90 protein;
b) contacting the cell with a test compound;
c) determining whether HSP70 protein expression is increased in the cell as compared to an untreated control, the expression of the HSP70 protein being an indication that the test compound may bind to the HSP90 protein.

According to one aspect of the present invention there is provided an assay for identifying compounds that modulate a neuronal apoptotic pathway, the assay comprising:
a) providing a cell expressing HSP90 protein;
b) contacting the cell with a test compound;
c) determining whether HSP70 protein expression is increased in the cell as compared to an untreated control, the expression of the HSP70 protein being an indication that the test compound may bind to the HSP90 protein.

### 2. Probes

The present invention provides for the use of compounds represented by either Formula I (probes). Probes of Formula 1 include identical substituents when compared to the compounds, or their pharmaceutically acceptable salts, of WO 03/051,890 A1 and WO 2004/111,060 A1 with the exception of the detectable labels or the affinity tags. Compounds and probes of the present invention can be synthesized using the chemistry or adaptations thereof that are disclosed in WO 03/051,890 A1 and WO 2004/111,060 A1.

According to one subset of the probes of Formula 1, there is provided the probe of the following formula:

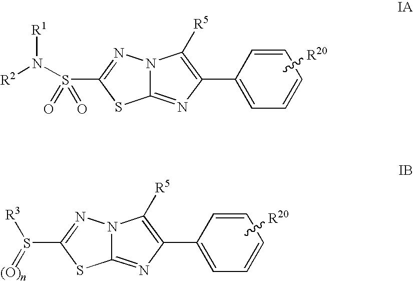

or a salt thereof,
wherein:
n is 1 or 2;
m is 1 to 20;
Y is NH, O or S;
R¹ and R² are independently selected from:
   1) H, or
   2) C₁-C₆ alkyl;
R³ is:
   1) C₁-C₆ alkyl,
   2) haloalkyl,
   3) aryl, or
   4) heteroaryl,
wherein the alkyl is optionally substituted with one or more R¹⁵ substituents; and the aryl and heteroaryl are optionally substituted with one or more R²⁰ substituents;
R⁵ is
   1) H, or
   2) halogen;
R⁷ is
   1) H,
   2) haloalkyl,
   3) C₁-C₆ alkyl,
   4) aryl,
   5) heteroaryl or
   6) heterocyclyl,
      wherein the alkyl is optionally substituted with one or more R¹⁵ substituents; and wherein the aryl, heteroaryl and heterocyclyl is optionally substituted with one or more R²⁰ substituents;
R¹⁰ is
   1) C₁-C₆ alkyl,
   2) C₃-C₇ cycloalkyl,
   3) haloalkyl,
   4) C₂-C₆ alkenyl,
   5) C₂-C₆ alkynyl,
   6) C₅-C₇ cycloalkenyl,
   7) aryl,
   8) heteroaryl, or
   9) heterocyclyl,
      wherein the alkyl, cycloalkyl, alkenyl, alkynyl, cycloalkenyl are optionally substituted with one or more R¹⁵ substituents, and the aryl, heteroaryl, and heterocyclyl are optionally substituted with one or more R²⁰ substituents;
R¹¹ and R¹² are independently selected from:
   1) H,
   2) C₁-C₆ alkyl,
   3) C₃-C₇ cycloalkyl,
   4) haloalkyl,
   5) aryl,
   6) heteroaryl,
   7) heterocyclyl,
   8) C(O) —C₁-C₆ alkyl,
   9) C(O)C₃-C₇ cycloalkyl
   10) C(O)-aryl,
   11) C(O)-heteroaryl,
   12) C(O)-heterocyclyl,
   13) C(O)Y—C₁-C₆ alkyl
   14) C(O)Y—C₃-C₇ cycloalkyl
   15) C(O)Y-aryl,
   16) C(O)Y-heteroaryl, or
   17) C(O)Y-heterocyclyl,
      wherein the alkyl and the cycloalkyl are optionally substituted with one or more R¹⁵ substituents, and the aryl, heteroaryl, and heterocyclyl are optionally substituted with one or more R²⁰ substituents;
      or R¹¹ and R¹² together with the nitrogen atom to which they are bonded form a five, six or seven membered heterocyclic ring optionally substituted with one or more R²⁰ substituents;
R¹⁴ is
   1) H, or
   2) C₁-C₆ alkyl;
R¹⁵ is
   1) NO₂,
   2) CN,
   3) halogen,
   4) C₃-C₇ cycloalkyl,
   5) haloalkyl,
   6) aryl,
   7) heteroaryl,
   8) heterocyclyl,
   9) OR⁷,
   10) S(O)ₙR¹⁰,
   11) NR¹¹R¹²,
   12) CHO,
   13) C(O)R¹⁰,
   14) CO₂R¹⁴,
   15) CONR¹¹R¹², or
   16) S(O)ₙNR¹¹R¹²,
      wherein the aryl and heteroaryl are optionally substituted with one or more R²⁵ substituents;
R²⁰ is
   1) NO₂,
   2) CN,
   3) N₃,
   4) B(OR¹⁴)₂,
   5) adamantyl,
   6) halogen,
   7) C₁-C₆ alkyl,
   8) C₂-C₆ alkenyl,
   9) C₂-C₄ alkynyl,
   10) C₃-C₇ cycloalkyl,
   11) C₃-C₇ cycloalkenyl,
   12) aryl,
   13) heteroaryl,
   14) heterocyclyl,
   15) haloalkyl,
   16) OR⁷
   17) SR⁷
   18) S(O)ₙR¹⁰,
   19) NR¹¹R¹²,
   20) CHO,
   21) C(O)R¹⁰,
   22) C(O)OR¹⁴
   23) CONR¹¹R¹²,
   24) S(O)ₙNR¹¹R¹²,
   25) NR¹⁴C(O)R¹⁰
   26) NR¹⁴S(O)₂R¹⁰,
   27) OC(O)R¹⁰,
   28) SC(O)R¹⁰,
   29) —O—(C₁-C₆alkyl-O)ₘR¹⁴, or
   30) —O—(C₁-C₆ alkyl-O)ₘ—C₁-C₆ alkyl-OR¹⁴,
      wherein the alkyl and the cycloalkyl are optionally substituted with one or more R¹⁵ substituents; and wherein the aryl, heteroaryl and the heterocyclyl are optionally substituted with one or more R²⁵ substituents;
R²⁵ is
   1) halogen,
   2) NO₂,
   3) CN,
   4) B(OR¹⁴)₂,
   5) haloalkyl,
   6) C₁-C₆ alkyl,
   7) C₂-C₆ alkenyl,
   8) C₂-C₄ alkynyl,
   9) OR⁷,
   10) SR⁷
   11) NR¹¹R¹²
   12) CHO,
   13) C(O)R¹⁰,
   14) C(O)OR¹⁴,
   15) CONR¹¹R¹²,
   16) S(O)₂R¹⁰,
   17) S(O)₂NR¹¹R¹²,
   18) NR¹⁴COR¹⁰,
   19) NR¹⁴S(O)₂R¹⁰,
   20) OC(O)R¹⁰, or
   21) SC(O)R¹⁰;
      wherein the probe comprises a detectable label or an affinity tag attached to any suitable position, and wherein the probe binds to HSP90 protein and is capable of being displaced by an inhibitor of the apoptotic JNK signaling pathway.

In one subset of the aforesaid probes, there is provided probes of the following formula:

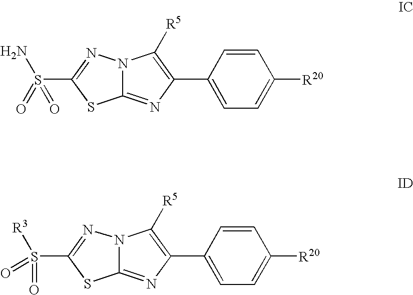

or a salt thereof,
wherein:
n is 1 or 2;
m is 1 to 20;
Y is NH, O or S;
R³ is C₁-C₆ alkyl optionally substituted with one or more R¹⁵ substituents;
R⁵ is
   1) H, or
   2) halogen;
R⁷ is
   1) H,
   2) haloalkyl,
   3) C₁-C₆ alkyl,
   4) aryl,
   5) heteroaryl or
   6) heterocyclyl,
      wherein the alkyl is optionally substituted with one or more R¹⁵ substituents; and wherein the aryl, heteroaryl and heterocyclyl is optionally substituted with one or more R²⁰ substituents;
R¹⁰ is
   1) C₁-C₆ alkyl,
   2) C₃-C₇ cycloalkyl,
   3) haloalkyl,
   4) C₂-C₆ alkenyl,
   5) C₂-C₆ alkynyl,
   6) C₅-C₇ cycloalkenyl,
   7) aryl,
   8) heteroaryl, or
   9) heterocyclyl,
      wherein the alkyl, cycloalkyl, alkenyl, alkynyl, cycloalkenyl are optionally substituted with one or more R¹⁵ substituents, and the aryl, heteroaryl, and heterocyclyl are optionally substituted with one or more R²⁰ substituents;
R¹¹ and R¹² are independently selected from:
   1) H,
   2) C₁-C₆ alkyl,
   3) C₃-C₇ cycloalkyl,
   4) haloalkyl,
   5) aryl,
   6) heteroaryl,
   7) heterocyclyl,
   8) C(O)—C₁-C₆ alkyl,
   9) C(O)—C₃-C₇ cycloalkyl
   10) C(O)-aryl,
   11) C(O)-heteroaryl,
   12) C(O)-heterocyclyl,
   13) C(O)Y—C₁-C₆ alkyl
   14) C(O)Y—C₃-C₇ cycloalkyl
   15) C(O)Y-aryl,
   16) C(O)Y-heteroaryl, or
   17) C(O)Y-heterocyclyl,
      wherein the alkyl and the cycloalkyl are optionally substituted with one or more R¹⁵ substituents, and the aryl, heteroaryl, and heterocyclyl are optionally substituted with one or more R²⁰ substituents;
R¹⁴ is
   1) H, or
   2) C₁-C₆ alkyl;
R¹⁵ is
   1) NO₂,
   2) CN,
   3) halogen,
   4) C₃-C₇ cycloalkyl,
   5) haloalkyl,
   6) aryl,
   7) heteroaryl,
   8) heterocyclyl,
   9) OR⁷,
   10) S(O)ₙR¹⁰,
   11) NR¹¹R¹²
   12) CHO,
   13) C(O)R¹⁰,
   14) CO₂R¹⁴,
   15) CONR¹¹R¹² or
   16) S(O)ₙNR¹¹R¹²,
      wherein the aryl and heteroaryl are optionally substituted with one or more R²⁵ substituents;
R²⁰ is
   1) NO₂,
   2) CN,
   3) N₃,
   4) B(OR¹⁴)₂,
   5) adamantyl,
   6) halogen,
   7) C₁-C₆ alkyl,
   8) C₂-C₆ alkenyl,
   9) C₂-C₄ alkynyl,
   10) C₃-C₇ cycloalkyl,
   11) C₃-C₇ cycloalkenyl,
   12) aryl,
   13) heteroaryl,
   14) heterocyclyl,
   15) haloalkyl,
   16) OR⁷,
   17) SR⁷,
   18) S(O)ᵣR¹⁰,
   19) NR¹¹R¹²,
   20) CHO,
   21) C(O)R¹⁰,
   22) C(O)OR¹⁴,
   23) CONR¹¹R¹²,
   24) S(O)ₙNR¹¹R¹²
   25) NR¹⁴C(O)R¹⁰,
   26) NR¹⁴S(O)₂R¹⁰,
   27) OC(O)R¹⁰,
   28) SC(O)R¹⁰,
   29) —O—(C₁-C₆alkyl-O)ₘR¹⁴, or
   30) —O—(C₁-C₆ alkyl-O)ₘ—C₁-C₆ alkyl-OR¹⁴,
      wherein the alkyl and the cycloalkyl are optionally substituted with one or more R¹⁵ substituents; and wherein the aryl, heteroaryl and the heterocyclyl are optionally substituted with one or more R²⁵ substituents;

### R²⁵ is

1) halogen,
2) NO₂,
3) CN,
4) B(OR¹⁴)₂,
5) haloalkyl,
6) C₁-C₆ alkyl,
7) C₂-C₆ alkenyl,
8) C₂-C₄ alkynyl,
9) OR⁷,
10) SR⁷
11) NR¹¹R¹²,
12) CHO,
13) C(O)R¹⁰,
14) C(O)OR¹⁴,
15) CONR¹¹R¹²,
16) S(O)₂R¹⁰,
17) S(O)₂NR¹¹R¹²,
18) NR¹⁴COR¹⁰,
19) NR¹⁴S(O)₂R¹⁰,
20) OC(O)R¹⁰, or
21) SC(O)R¹⁰;
   wherein the probe is either:
a) labeled with a radioactive isotope at any suitable position;
b) linked to a detectable moiety by a suitable linker at R³ or R²⁰;
c) linked to an affinity tag at any suitable position; or
d) linked to a polymer support;
   and wherein the probe binds to HSP90 protein and is capable of being displaced by an inhibitor, and inhibiting an apoptotic JNK signaling pathway.

The probes of the present invention are typically labeled with a radioactive label at any suitable position. As will be readily understood by a person skilled in the art, a radioactive label can be incorporated within the probe of Formula I at any suitable position. For example a ³H or ¹⁴C isotope can replace any hydrogen or carbon present in the molecule. Similarly, a ¹²⁵I isotope can be substituted on any aromatic ring or can replace any hydrogen atom.

As used herein, the term “detectable label” is intended to mean a group that may be linked to a compound of the present invention to produce a probe or to HSP90 protein, such that when the probe is associated with the HSP90, the label allows either direct or indirect recognition of the probe so that it may be detected, measured and quantified. Examples of such “labels” are intended to include, but are not limited to, fluorescent labels (for example fluorescein, Oregon green, dansyl, rhodamine, tetra-methyl rhodamine, Texas-red, phycoerythrin BODIPY.FL, BODIPY 493/503 or Eu³⁺), chemiluminescent labels (for example luciferase), calorimetric labels, enzymatic markers, particles such as magnetic particles, radioactive isotopes (for example ³H, ¹⁴C, ¹²⁵I) and affinity tags for example biotin. The labels described can be attached to the probe or to the HSP90 protein using well known methods.

As used herein, the term “affinity tag” is intended to mean a ligand or group, which is linked to either a compound of the present invention or to HSP90 protein to allow another compound to be extracted from a solution to which the ligand or group is attached. Examples of such ligands include biotin or a derivative thereof, a polyhistidine peptide, an amylose sugar moiety or a defined epitope recognizable by a specific antibody. The affinity tags described can be attached to the probe or to the HSP90 using well known methods.

In some examples, only one of the components is labeled. For example, the HSP90 protein may be labeled at various amino acid residues using ¹²⁵I, fluorophores or with TEMPO derivatives, by methods known in the art. Alternatively, more than one component may be labeled with different labels; using ¹²⁵I for the HSP90 protein, for example, and a fluorophor for the compound.

Specifically, according to one aspect of the present invention, there is provided the following probes:

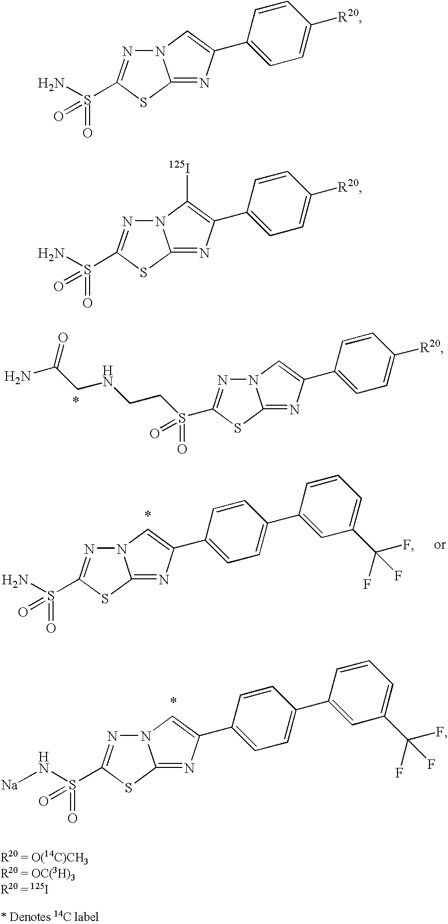

wherein R²⁰ is as defined herein.

According to an alternative aspect of the present invention, there is provided the following probes:

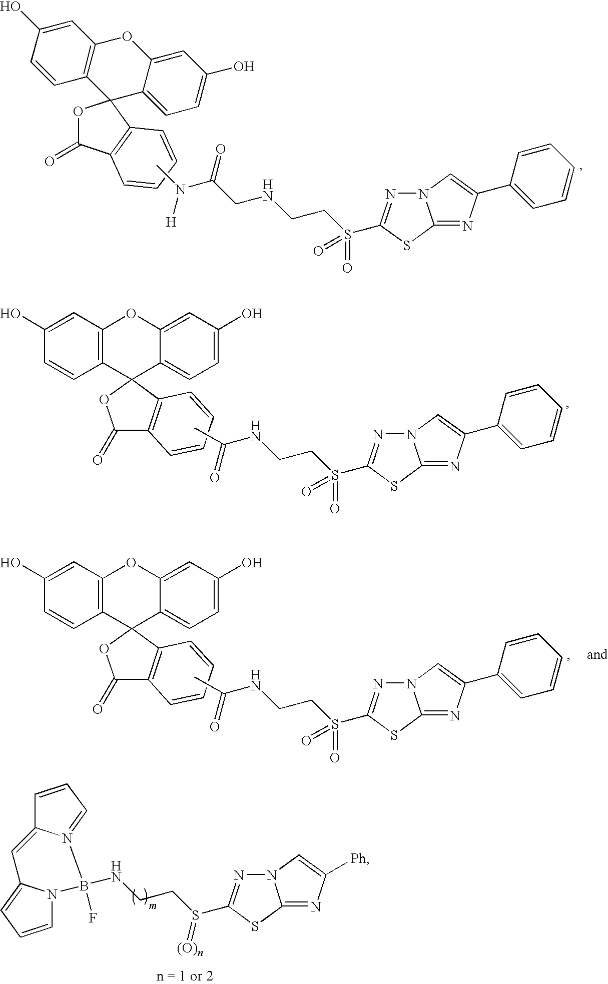

According to an alternative aspect of the present invention, there is provided the following probes including compounds 1 to 6 and 10 to 20.

According to an alternative aspect of the present invention, there is provided a polymer supported affinity probe characterized by the conjugation of compound 6 with a functionalized polymeric support.

According to an alternative aspect of the present invention, there is provided the following probes including compound 7.

The probes of the invention may also be used as competitors to screen for additional drug candidates. “Candidate bioactive agent” or “drug candidate” or “test compound” or grammatical equivalents thereof describe any molecule, for example, protein, oligopeptide, small organic molecule, polysaccharide, polynucleotide, and the like, to be tested for bioactivity. They may be capable of directly or indirectly altering the expression of HSP70 and/or the apoptotic JNK signaling pathway. In other cases, alteration of HSP90 protein binding and/or activity may be screened. In the case where HSP90 protein binding or activity is screened, one example might include the exclusion of molecules already known to bind to that particular protein. Alternately, these known compounds may be used as positive controls to validate the binding assay or further correlate the locus of HSP90:compound binding. More specifically, compounds known to bind to the N-terminal HSP90 APT-binding site may be used to confirm that the test compounds are not bind to the N-terminal HSP90 APT-binding site.

Test compounds can encompass numerous chemical classes, though typically they are organic molecules, such as small organic compounds having a molecular weight of more than 100 and less than about 2,500 daltons. Test compounds comprise functional groups necessary for structural interaction with proteins, particularly hydrogen bonding and lipophilic binding, and typically include an amine, carbonyl, hydroxyl, ether, or carboxyl group, preferably at least two of the functional chemical groups. The test compounds often comprise cyclical carbon or heterocyclic structures and/or aromatic or polyaromatic structures substituted with one or more of the above functional groups. Test compounds are also found among biomolecules including peptides, saccharides, fatty acids, steroids, purines, pyrimidines, derivatives, structural analogs or combinations thereof.

Test compounds can be obtained from a wide variety of sources including libraries of synthetic or natural compounds. For example, numerous methods are available for random and directed synthesis of a wide variety of organic compounds and biomolecules. Alternatively, libraries of natural product compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs. Libraries of small molecular weight compounds, generally referred to as fragment based libraries, are routinely screened at high concentrations in order to identify low affinity ligands. Synthetic modifications or linking of one or more low affinity ligands often leads to significant increases in protein binding by these new entities. These methods are included within the scope of this invention.

### Imidazo[2,1-b]-1,3,4-thiadiazole-2-sulfonamides and imidazo[2,1-b]-1,3,4-thiadiazole-2-sulfones and Sulfoxides Inhibit NGF-Withdrawal, Taxol or Cisplatin Induced Death in SCG Neurons

Survival of neonatal primary sympathetic neurons is dependent on trophic support provided by Neuronal Growth Factor (NGF) (reviewed in (Young et al., 2004)). The inventors have previously demonstrated that imidazo[2,1-b]-1,3,4-thiadiazole-2-sulfonamides, and imidazo[2,1-b]-1,3,4-thiadiazole-2-sulfones and imidazo[2,1-b]-1,3,4-thiadiazole-2-sulfoxides, including compounds 1, 2, 5, 6, and 10 to 20 of the present invention, inhibit NGF withdrawal induced death in SCG neurons. Similarly, the inventors previously demonstrated that compounds 1, 2, 5, 6, and 10 to 20 were shown to inhibit Taxol or cisplatin induced apoptosis in SCG neurons. The results presented herein demonstrate that this class of compounds shares a novel mechanism of action involving binding of the compounds to HSP90 protein, and that the mechanism of action of NGF withdrawal and Taxol or cisplatin treatment converge on JNK mediated apoptotic signaling.

Compound 1 Inhibits p75NTR- or NRAGE-Induced Apoptosis of PC12 Cells

Adenoviral-mediated over expression of p75NTR, or its cytosolic interactor, NRAGE, leads to extensive JNK-dependent apoptosis of PC 2 cells and primary cortical neurons (Bhakar et al., 2003; Salehi et al., 2000; Salehi et al., 2002). Because the signaling pathways that lead to p75NTR- or NRAGE-induced cell death have been unambiguously established in PC12 cells, this system was employed to determine the mechanism of action of compound 1. We previously produced an adenovirus that drives NRAGE expression via a doxycycline inducible element (constitutive NRAGE expression is cytotoxic—see (Salehi et al., 2002) for details) and for the experiments described below, a PC12 subline (PC12rtTA) that stably expresses the doxycycline activated transcription factor, rtTA, was used.

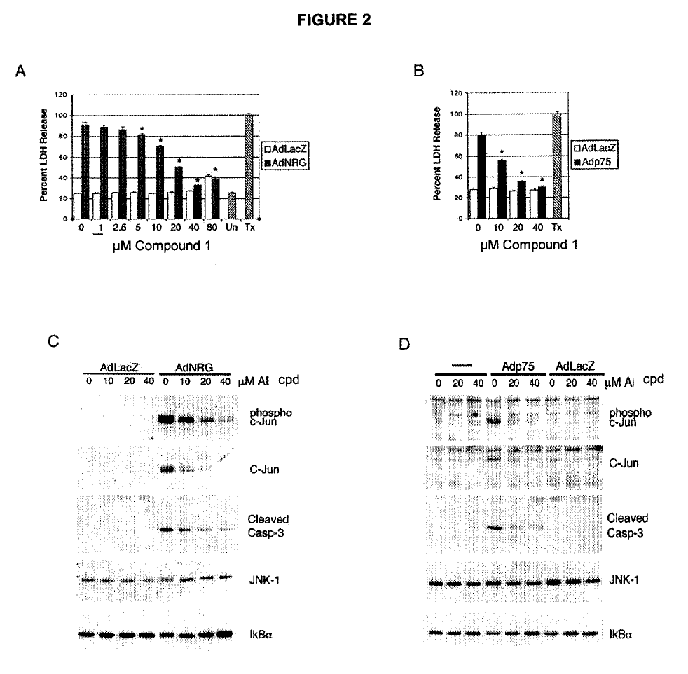

PC12^{rtTA} were infected with adenoviruses encoding p75NTR (Adp75), NRAGE (AdNRG), or as a control, LacZ (AdLacZ), and were concurrently exposed to increasing concentrations of compound 1 for a period of 40 hours. Cell death was assessed using an LDH release assay. Overexpression of NRAGE or p75NTR led to extensive death of PC12^{tTA} cells, which was strongly attenuated by co-treatment with compound 1 in a dose-dependent manner (FIGS. 2A, B). Treatment with 40 μM compound 1 reduced p75NTR- or NRAGE-induced cell death by greater than 90%. At the highest concentration tested (80 μM), the compound effectively inhibited apoptosis, but also exerted a slight toxic effect in cells infected with the LacZ control. These results indicated that the PC12^{tTA} overexpression paradigm provided a convenient and biochemically tractable system for analyzing the mechanism of action of compound 1.

Compound 1 Inhibits p75NTR or NRAGE Mediated JNK Activation

Activation of JNK activity is necessary for the induction of p75NTR- or NRAGE-initiated caspase cleavage and cell death (Bhakar et al., 2003; Salehi et al., 2002). Therefore, the effect of compound 1 on the activity of JNK was assessed by analyzing alterations in the phosphorylation level of the JNK target, c-Jun, and the cleavage status of caspase-3. FIG. 2C shows immunoblots of lysates of PC12rtTA cells that were infected with AdNRG, or the control virus, AdLacZ, in the presence of increasing concentrations of compound 1. In the absence of compound 1, NRAGE expression resulted in robust c-Jun phosphorylation and caspase-3 cleavage. Levels of c-Jun protein were also increased, presumably due to the auto-activation of c-Jun transcription by phosphorylated c-Jun protein (Angel et al., 1988). Treatment with compound 1 strongly attenuated each of these effects; a significant decrease in c-Jun phosphorylation and caspase-3 cleavage was detectable at 10 μM compound 1 and was virtually complete at 40 μM. FIG. 2D shows that similar results were obtained in cells infected with Adp75 and treated with increasing concentrations of compound 1. Thus, the suppression of JNK signaling and caspase-3 cleavage mediated by compound 1 occurred with a dose-dependency similar to the anti-apoptotic activity of compound 1.

### Compound 1 Inhibits PC12 Cell Death Induced by Paclitaxel and Cisplatin

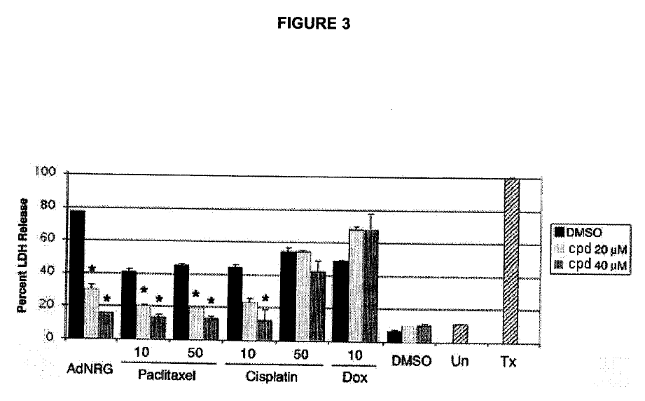

The ability of compound 1 to inhibit apoptosis in response to a variety of other insults, including the DNA damaging agent, cisplatin, the microtubule disruptor, paclitaxel, and doxorubicin, a topoisomerase inhibitor was investigated. Previous studies demonstrate that cell death induced by paclitaxel is dependent on JNK activation (Figueroa-Masot et al., 2001; Lee et al., 1998; Srivastava et al., 1999) and FIG. 3 shows that apoptosis induced by low (10 μM) and high (50 μM) concentrations of paclitaxel are effectively reduced by compound 1. Cisplatin-induced apoptosis has been reported to be JNK-dependent when used at low concentrations (10 μM), but JNK-independent at concentrations >25 μM (Sanchez-Perez and Perona, 1999) and consistent with this, compound 1 protected cells exposed to 10 μM, but not 50 μM, cisplatin (FIG. 3). Doxorubicin-induced apoptosis is JNK-independent, and compound 1 treatment did not offer any protection against doxorubicin-induced cell death. Collectively, these results are consistent with the hypothesis that compound 1 inhibits apoptosis by blocking the JNK pathway.

### Compound 1 Induces Expression of HSP70

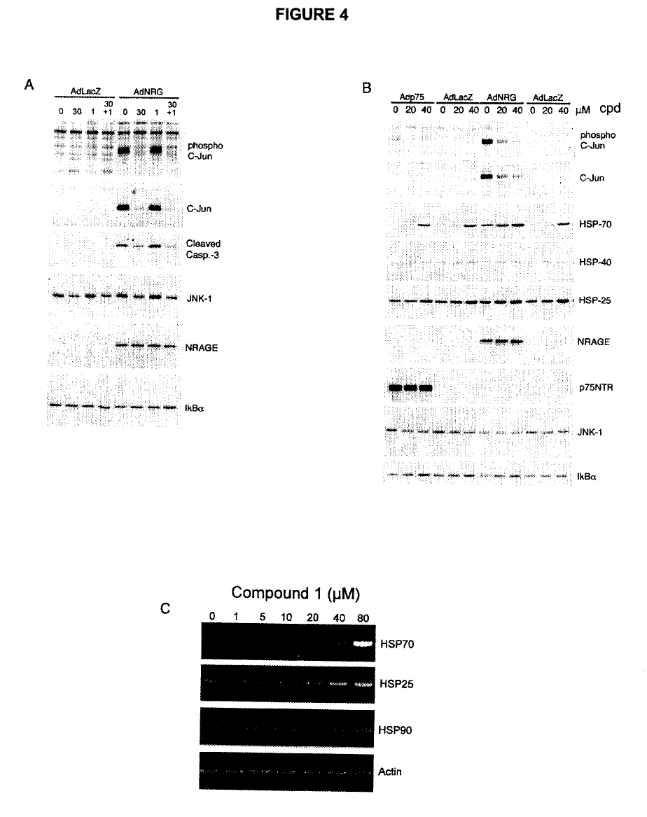

The structure of compound 1 does not resemble previously identified kinase inhibitors, and compound 1 has no effect on JNK activity in vitro (data not shown). The conclusion that compound 1 does not directly suppress JNK activity was supported by the observation that the JNK inhibitory effects of compound 1 occurred in cells subjected to extended (i.e.; 24 hour), but not short term (i.e.; 1 hr hour), incubation with the compound (FIG. 4A). These results suggested that compound 1 suppresses apoptotic signaling through alternate means, possibly by enhancing transcription and translation of an endogenous factor which inhibits JNK activity. Several studies have established that heat shock protein 70 (HSP70) can bind and inhibit JNK (Gabai et al., 2002; Gabai et al., 2000; Parcellier et al., 2003; Park et al., 2001; Yaglom et al., 1999) and the possibility that compound 1 promotes HSP70 production and thereby inhibits the JNK signaling pathway was therefore explored.

To address this, SCG neurons were treated with compound 1 for 18 hours then lysed and assessed for HSP70 levels by immunoblot. Uninfected PC12rtTA cells were treated in a similar manner. In both cases compound 1 treatment results in a large increase in cellular HSP70 levels, as shown in FIG. 4B for uninfected PC12rtTA cells. To test if the accumulation of HSP70 induced by compound 1 reflected increased steady state levels of its mRNA, RNA was isolated from PC12 cells treated with increasing concentrations of compound 1 for 18 hours and the cell lysates subjected to semi-quantitative rtPCR. FIG. 4C shows that compound 1 treatment increased levels of HSP25 and HSP70 mRNA, but had no effect on HSP90 or actin mRNA levels. HSP protein levels were also assessed in PC12rtTA cells infected with AdLacZ, Adp75, or AdNRG, and at the same time, exposed to compound 1. FIG. 4D shows that treatment of PC12rtTA cells with increasing concentrations of compound 1 resulted in a robust increase in HSP70 and HSP25, but not HSP40, in cells infected with either LacZ or with Adp75. Cells infected with AdNRG showed enhanced expression of HSP70 even in the absence of compound 1. However, treatment of cells infected with AdNRG pushed HSP70 levels substantially higher that those observed with AdNRG alone.

The induction of HSP70 was investigated in vivo by the treatment of rats with a single dose of Na-19 (SC, 30 mg/kg). HSP70 induction was demonstrated in excised and homogenized sciatic nerve. Full body radiography with [¹⁴C]—Na-19 demonstrated significant, time dependent, radioactivity in sciatic nerve, suggesting that the observed HSP70 induction was drug related.

Collectively, these data demonstrate that treatment of SCG neurons or PC12s with compounds 1 protects them from various apoptotic insults and that this neuroprotection is associated to the induction of HSP70 protein both in vitro and in vivo.

### Transcriptional Regulation of HSP70 and HSP25, but not HSP40, is Mediated by the Heat Shock Factor 1 (HSF1) Transcription Factor

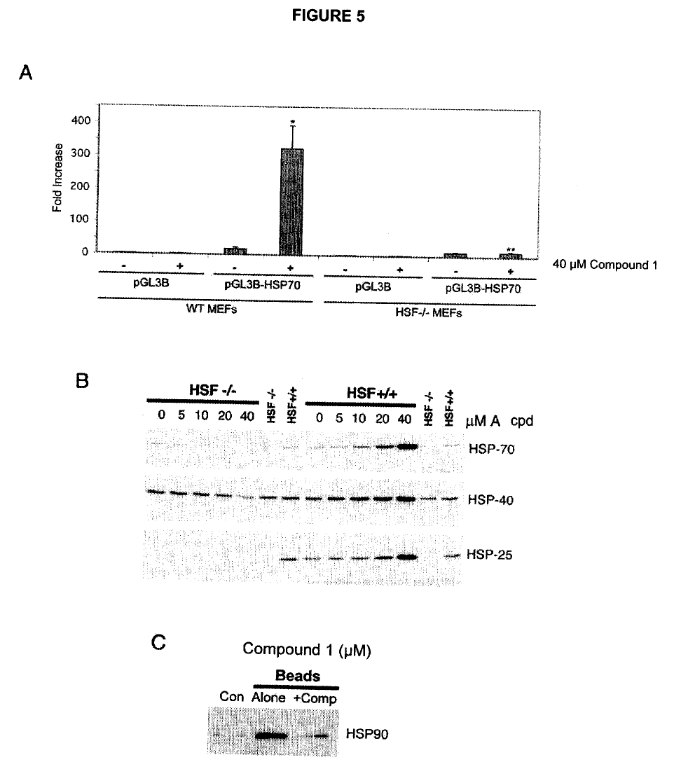

In unstimulated cells, HSF1 is maintained in an inactive, latent state through an interaction with its binding/chaperone partner, HSP90. Induction of cell stress results in the interaction of HSP90 with misfolded proteins and disrupts its association with HSF1, allowing it to bind and activate HSP70 and HSP25 promoters (Sanchez-Perez and Perona, 1999). The specific effect of compound 1 on HSP25 and HSP70 production suggested that compound 1 binds HSP90, causing it to release HSF1 which then binds and activates HSP70 and HSP25 promoters. The activity of HSP70 transcriptional reporter constructs transfected into PC12 cells were found to be stimulated by compound 1. To specifically address whether HSF1 plays a role in this response, the effects of compound 1 on mouse embryonic fibroblasts (MEFs) derived from either wild type or HSF1 null mice were examined (McMillan et al., 1998). FIG. 5A shows that transcriptional activity of the HSP70 promoter reporter construct was strongly induced by compound 1 in wild-type MEFs, but remained at baseline in MEFs lacking HSF1. Expression of endogenous HSP proteins showed corresponding changes in the MEFs, with strong induction of HSP70 and HSP25 in the wild type cells but not in MEFs lacking HSF1 (FIG. 5B).

These data indicate that compound 1 mediates activation of HSP proteins through an HSF1-dependent pathway, and are consistent with the hypothesis that compound 1 directly binds HSP90.

### Imidazo[2,1-b]-1,3,4-thiadiazole-2-sulfonamides and imidazo[2,1-b]-1,3,4-thiadiazole-2-sulfones and Sulfoxides Bind to HSP90

Two probes were prepared in an effort to identify proteins which bind to this class of compounds. The syntheses of compounds 6 and 7 have been previously disclosed in WO 2004/111,060 A1. The compounds 6 and 7 were shown to be useful probes in the identification of target proteins for imidazo[2,1-b]-1,3,4-thiadiazole-2-sulfonamides and imidazo[2,1-b]-1,3,4-thiadiazole-2-sulfones and sulfoxides.

Compound 6 was coupled with Affi-Gel 10 beads (BioRad) and used as an affinity reagent in pulldown experiments. Beads were incubated either with whole cell lysates or with purified HSP90, washed extensively, and bound proteins were then eluted and analyzed by SDS-PAGE and immunoblotting. FIG. 5C shows that compound 6-beads, but not control beads, bound HSP90 and that this interaction was strongly attenuated by incubation with excess unbound compound 1. This probe in this reaction was a imidazo[2,1-b]-1,3,4-thiadiazole-2-sulfoxide-bead. The binding of the compound 6-bead conjugate with HSP90 was augmented by the presence of compound 1, a imidazo[2,1-b]-1,3,4-thiadiazole-2-sulfonamide, suggesting that these two classes of compounds both bind to HSP90.

HSP90 was also identified using the photoaffinity-biotinylated probe, compound 7. PC12 protein extracts were separated by SDS page. SDS was removed by incubation in 20% isopropanol and proteins were renatured by incubation in DTT and guanidine HCl. The gel was then incubated for 3 hours with 2 uM compound 7 and then photoactivated for 30 minutes using a UV crosslinker. Proteins were then transferred to nitrocellulose and biotin was detected with HRP-conjugated strepavidin. A band was observed which corresponded to an approximately 90 kDal protein, consistent with binding to HSP90.

Collectively, these data indicate that imidazo[2,1-b]-1,3,4-thiadiazole-2-sulfonamides and imidazo[2,1-b]-1,3,4-thiadiazole-2-sulfones and sulfoxides bind to HSP90, thereby facilitating HSF1-dependent expression of HSP70 and HSP25.

Compound:bead conjugates prepared using imidazo[2,1-b]-1,3,4-thiadiazole-2-sulfoxides and sulfones will be useful in assays for the identification of test compounds (library compounds) which bind HSP90 in a similar manner to compounds of formula I. For example, compound:bead conjugates can be first treated with labeled-HSP90 protein. After the addition of test compounds and incubation for a given time, the compound:bead:HSP90 complex is washed, and released labeled-HSP90 is quantified by either quantifying the amount of labeled-HSP90 remaining in the complex, or by quantifying the amount of released labeled-HSP90 in the washings. Changes in the signal from baseline would identify test compounds which were able to disrupt the compound:bead:HSP90 complex. HSP90 protein may be labeled, for example, with fluorescent labels or with radiolabels.

### Inhibition of JNK by imidazo[2,1-b]-1,3,4-thiadiazole-2-sulfonamides and imidazo[2,1-b]-1,3,4-thiadiazole-2-sulfones and Sulfoxides

An elevation in JNK activity is observed when PC12 cells are subjected to various stresses, including the treatment with staurosporine (Yoshazumi, M., et al., British Journal of Pharmacology 2002, 136, 1023). In order to reliably assay the ability of this class of compounds to inhibit JNK PC12s were treated with compound plus staurosporine and JNK activation was measured by c-Jun phosphorylation.

PC12 cells were plated at a density of 2.5×10⁶ cells per well in 6-well plates. The following day, cells were pretreated with compound at the desired concentrations. After 4 hours they were treated with 0.5 μM staurosporine and incubated overnight. Cells were lysed and the proteins in each sample were quantified. A selective IP was performed by incubating 300 μg of each cell lysate sample with 20 μL of immobilized c-Jun fusion protein bead slurry on a rotating wheel overnight at 4° C. IP pellets were washed and incubated in a kinase buffer containing cold ATP. c-Jun phosphorylation in each sample was analyzed using the phospho c-Jun (Ser63) antibody by Western blotting.

A series of imidazo[2,1-b]-1,3,4-thiadiazole-2-sulfonamides and imidazo[2,1-b]-1,3,4-thiadiazole-2-sulfones and sulfoxides were tested for their ability to inhibit JNK as described above. Compounds 10 through 17 were shown to inhibit staurosporine induced JNK activity at drug concentrations between 20 and 50 uM.

### Compound 1 Inhibition of JNK Activity is Mediated by the Induction of HSP70

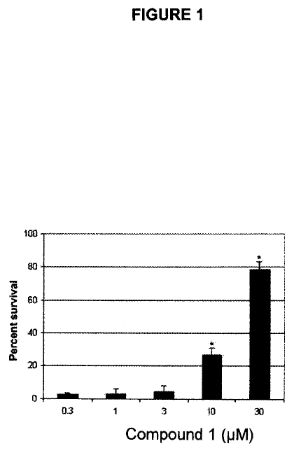

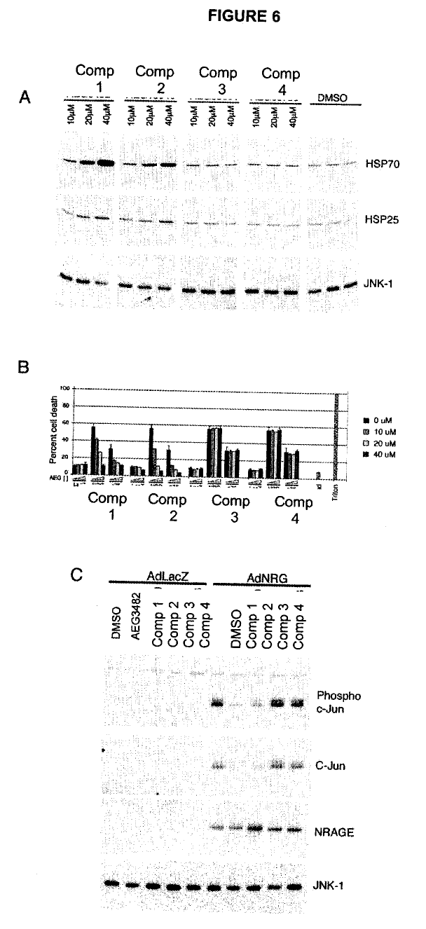

Previous studies have shown that HSP70, but not HSP25, is capable of blocking JNK activation (Gabai et al., 2002; Gabai et al., 2000; Parcellier et al., 2003; Park et al., 2001; Yaglom et al., 1999). The functional interaction between compound 1, HSP70 induction and JNK pathway inhibition were examined by comparing the cellular effects of compounds 1, 2, 3, and 4 (FIG. 1). Compound 1 and 2 induced HSP70 production, whereas compounds 3 and 4 failed to induce HSP70 (FIG. 6A). As stated above, compounds 1 and 2 protected SCG neurons from NGF withdrawal and protected PC12 cells from Adp75- or AdNRG-induced cell death (FIG. 6B) while reducing c-Jun phosphorylation (FIG. 6C). On the other hand, compounds 3 and 4 do not protect SCG neurons or PC12 cells from the above insults. Thus, using these four related compounds, a strong correlation emerged between the induction of HSP70, the inhibition of JNK activity, and cell death.

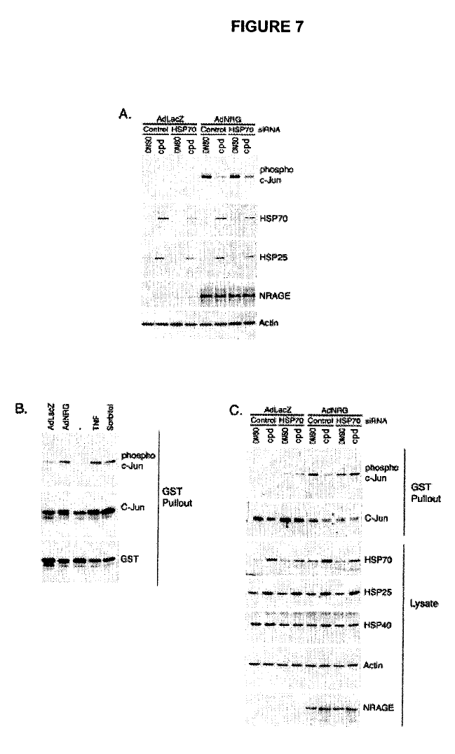

Loss of function experiments established a direct causal link between the induction of HSP70 by these compounds and their JNK inhibitory activity. For this, RNA interference directed against HSP70 was used to block its accumulation following treatment with compound 1. The expectation was that if induction of HSP70 by the compounds was required for JNK inhibition, then blocking the accumulation of HSP70 should reduce the JNK inhibitory effect. FIG. 7A shows that transfection with HSP70 siRNA modestly attenuated expression of HSP70 induced by compound 1. The relatively poor HSP70 knockdown likely reflects the low transfection efficiencies that can be achieved in PC12rtTA cells. Despite this, a small but consistent attenuation in the ability of compound 1 to block c-jun phosphorylation was observed in cells transfected with HSP70 RNAi, consistent with the hypothesis that HSP70 induction is required for this effect.

A causal link between HSP70 induction and JNK suppression, circumventing the problem of low-transfection efficiency of the PC12rtTA cells, was developed to assess JNK pathway activation specifically in the RNAi transfected subpopulation of PC12rtTA cells. Using a mammalian expression vector driving production of a fusion protein containing GST fused to amino acids 2-79 of c-Jun. We anticipated that the GST-c-Jun fusion protein would be readily recoverable from lysates and its phosphostatus would provide a sensitive read-out that reflects the level of JNK pathway activation in the subpopulation of cells which were transfected. Although overall transfection efficiencies are low in PC12rtTA cells, the GST-c-Jun fusion plasmid and the HSP70 siRNA would be efficiently co-transfected, thereby providing an accurate assessment of the effect of HSP70 depletion on JNK signaling. To validate the GST-c-Jun fusion protein as an in vivo reporter of JNK activity, PC12rtTA cells transfected with GST-c-Jun were treated with TNF or exposed to hyperosmotic shock, two stimuli commonly used to activate the JNK signaling pathway (Davis, 2000). GST-c-Jun was then recovered from lysates and its phosphostatus determined by immunoblotting. FIG. 7B shows that both TNF and hyperosmotic shock cause a dramatic increase in phosphorylation of GST-jun which is readily detected by immunoblot. Thus, the phosphostatus of the GST-jun fusion protein provided a sensitive read-out that reflects the level of JNK pathway activation in transfected cells.

HSP70 accumulation was required to block NRAGE-induced JNK activity. Compound 1 treatment itself significantly increased expression of the GST-c-Jun fusion construct, complicating the interpretation of results (data not shown). As an alternative, compound 2 was used since it robustly induced HSP70 production (see FIG. 6) without altering GST-Jun expression levels. FIG. 7C shows that in cells transfected with control siRNA, infection with AdNRG dramatically increased phosphorylation of GST-c-Jun, whereas compound 2 strongly inhibited this effect In cells transfected with RNAi directed against HSP70, the ability of compound 2 to block GST-jun phosphorylation was almost completely lost, indicating that HSP70 accumulation plays a crucial role in the JNK inhibition elicited by this series of compounds.

### Compound 1 and Geldanamyin Differ in Their Mechanism of Action

Geldanamycin is a benzoquinone ansamycin that binds to the ATP-binding pocket of HSP90 (reviewed in Goetz et al., 2003; Sreedhar et al., 2004; Workman, 2004). To determine if compound 1 also occupies the HSP90 binding pocket, we compared compound 1 and geldanamycin for their ability to block binding of HSP90 to gATP-Sepharose. Purified HSP90 readily binds gATP-Sepharose and is this strongly inhibited in the presence of free ATP. Geldanamyin also blocks association of HSP90 with gATP-Sepharose, consistent with previous results (Grenert et al., 1997); in contrast, compound 1 does not decrease the amount of HSP90 bound to gATP-Sepharose but rather increased their association.

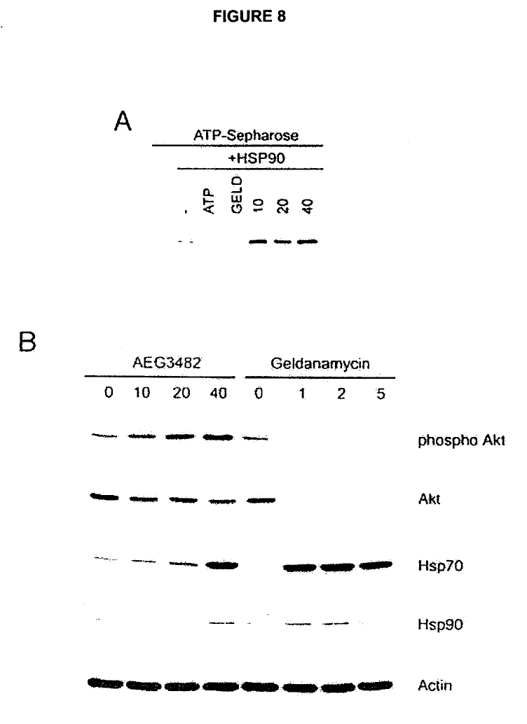

By occupying the ATP binding pocket, geldanamycin is thought to block the chaperone function of HSP90 and therefore reduces the stability and activity of HSP90 client proteins. We therefore compared the effects of compound 1 and geldanamycin on Akt, a pro-survival kinase that is an HSP90 client protein. FIG. 8B shows that in PC12 cells, geldanamycin treatment lead to a dramatic reduction in levels of total and phosphorylated Akt, consistent with previous results (Kim et al., 2003), whereas compound 1 significantly enhanced pAkt levels and reduced total Akt only slightly at the highest concentrations tested.

These results demonstrate that unlike previously reported HSP90 binding compounds, compound 1 does not bind the N-terminal ATP-binding site. Additionally, compound 1 augments the binding of HSP90 to ATP, as seen by increased binding of HSP90 to gATP-Sepharose in the presence of compound 1. Therefore, imidazo[2,1-b]-1,3,4-thiadiazole-2-sulfonamide such as compound 1, will demonstrate different biological effects as compared to compounds which bind the N-terminal ATP-binding site by augmenting the chaperoning properties of HSP90.

### Saturation Transfer Difference (STD) NMR Spectroscopy

STD NMR spectroscopy is a powerful tool that has been used to characterize the binding of various small molecules to proteins by quantifying NOE energy transfer from irradiated protein to test compounds (see Meyer and Peters, T. Angew. Chem. Int. Ed. 2003, 42, 864 for a review of current methods). These methods allow not only for the identification of compounds which bind to a particular protein, but also for the identification of specific binding moieties within a compound. STD NMR spectroscopy has been used identify test compounds from compound libraries which bind to a given protein by the identification of test compounds which display positive (Nuclear Overhauser effect) NOE between the protein and test compounds. Further, these methods can be used of quantify the binding kinetics between test compounds.

STD ¹H NMR spectroscopy was used to demonstrate binding of imidazo[2,1-b]-1,3,4-thiadiazole-2-sulfonamide and sulfoxide compounds to HSP90 using known pulse sequences. STD NMR experiments were performed at 19° C. on a Varian INNOVA 500 MHz spectrometer equipped with a triple resonance HCN cold probe. A 1D saturation transfer difference pulse sequence with internal subtraction via phase cycling was employed (Mayer and Meyer, JACS, 2001, 123, 6108-6117). Residual HDO signal was removed using a W5 WATERGATE pulse sequence, with a 150 ms interpulse delay (Liu et. al, J. Magn. Res., 1998, 132, 125). On resonance irradiation of the protein was performed at −0.5 ppm, with off-resonance irradiation at 34 ppm. The irradiation power was (g/2p)B₁=93 Hz applied through a train of 50 ms eburp1 pulses with a 1 ms delay between the pulses. The total presaturation time was 5.1 s. The relaxation delay was set to 0.1 s. A 30 ms spin-lock pulse with a strength of (g/2p)B₁=4600 Hz was used to eliminate background protein signals. The total number of scans was between 1536-2048, with a sweep width of 16 ppm. Spectra were multiplied by a 1 Hz exponential line-broadening function prior to Fourier transform.

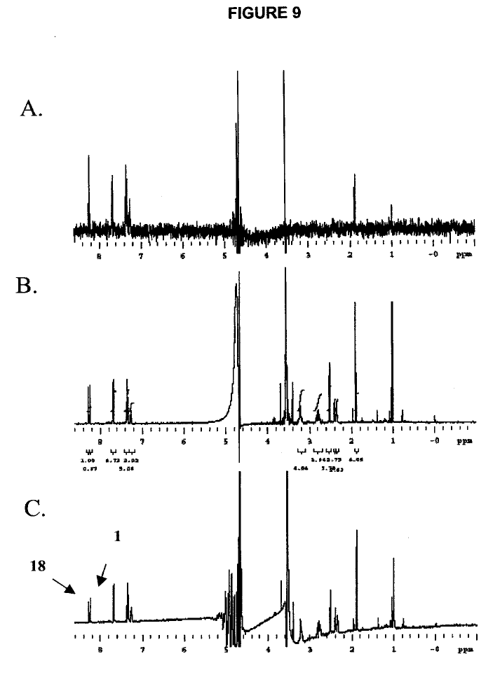

Positive protein to compound NOE saturation was observed in the STD ¹H NMR spectra of compounds 20 in the presence of HSP90 protein (Stressgen Bioreagents). The compound to protein ratio was fixed at approximately 100:1 in 20 mM sodium phosphate buffer (pH 6.7). Compound 20 was used as a reference compound to compare binding affinities of various compounds to HSP90 by comparing the relative intensity of NOE effects in the spectra of compounds 1 and 20 (see FIG. 9), compounds 2 and 20, compounds 5 and 20, compounds 13 and 20, and compounds 15 and 20 (near equamolar quantities of each) in the presence of HSP90 protein. Using the NOE signals for the imidazole proton, and comparing peak heights, the relative k_{d} of the compound pairs were compared (see Meyer and Peters, T. Angew. Chem. Int. Ed. 2003, 42, 864). The k_{d}s of the compound pairs ranged from 1:1 to 8:1. These differences in binding affinities correlated to the potency of each respective compound's ability to rescue SCG neurons from NGF withdrawal as previously reported.

This study demonstrates that both imidazo[2,1-b]-1,3,4-thiadiazole-2-sulfonamides and imidazo[2,1-b]-1,3,4-thiadiazole-2-sulfoxides bind to HSP90. Further, this defines the parameters for STD NMR spectroscopy based assays used to identify other ligands/compounds which bind to the same site on HSP90.

The use of STD NMR spectroscopy in the screening of compound using low affinity or fragment based approaches are well known in the art (for a review see Hujduk, P J. Mol. Interv. 2006, 6, 266-72).

Thus, the present invention also provides a method of screening compounds for binding to an HSP90 protein, the method comprising:
a) mixing at least one aqueous soluble test compound with the HSP90 protein to form a compound:HSP90 complex in an aqueous medium;
c) measuring a STD NMR spectrum of the test compound and the HSP90 protein mixture;
d) identifying a compound which demonstrates STD or resonance shift, compared to a control.

Imidazo[2,1-b]-1,3,4-thiadiazole-2-sulfonamides and imidazo[2,1-b]-1,3,4-thiadiazole-2-sulfoxides and sulfones probes of the instant invention may be used in competitive STD ¹H NMR spectroscopy assays to validate that the library compounds which bind HSP90 protein are binding in a similar manner to the probes. A measure of the relative k_{d}s of library compounds can be compared to that of the probe using the relative NOEs, as described above. Alternatively 2D STD ¹H NMR spectroscopic methods can be used to confirm that compounds are bind to the same site, as compounds which bind to the same site will demonstrate cross peaks in the 2D ¹H STD NMR spectrum. Further, ¹⁵N or ¹³C labeled probes may be used and their binding measured by STD ¹⁵N or ¹³C NMR spectroscopy.

Alternatively, the assay may use probes of the instant invention to identify library compounds which either positively or negatively augment the binding of the probe to HSP90, as described in the following competition binding assay.

Thus, the present invention also provides a competition binding assay for screening for compounds which bind to HSP90, the assay comprising:
a) contacting a probe of the present invention with an HSP90 protein to form a probe:HSP90 complex in an aqueous medium;
c) contacting the probe:HSP90 complex with at least one compound from a compound library to form a mixture;
d) measuring a STD NMR spectrum of the mixture;
e) identifying compounds which demonstrate STD or resonance shifts associated with the NMR resonances of the probe.

Many other variations may be envisioned as described in Meyer and Peters, T. Angew. Chem. Int. Ed. 2003, 42, 864 and Hujduk, P J. Mol. Interv. 2006, 6, 266-72, and references cited therein, incorporated herein by reference.

### DISCUSSION

The present study indicates that the anti-apoptotic activity of compound 1 and its analogs arises from their ability to block activation of the JNK pathway. Structurally, compound 1 does not resemble known kinase inhibitors, and its time course of action suggested that it may function by facilitating production of an endogenous JNK pathway antagonist. HSP70 is an endogenous inhibitor of JNK activity (Gabai et al., 2002; Gabai et al., 2000; Parcellier et al., 2003; Park et al., 2001; Yaglom et al., 1999) and our data show that compound 1 is a potent inducer of HSP70 production and demonstrate that HSP70 accumulation is required for the effect of compound 1 on JNK signaling. To our knowledge, this compound is the first shown to inhibit JNK activation through a mechanism involving induced production of HSP70.

HSPs are highly conserved proteins that are induced by a wide variety of chemical and physiological stimuli (reviewed in (Sreedhar and Csermely, 2004)). It is well established that HSPs play crucial protective roles in stress responses and that they can suppress apoptosis induced by heat shock, chemotherapeutic agents, nutrient withdrawal, ionizing radiation or TNF (Sreedhar and Csermely, 2004). Induction of HSPs with sublethal stresses gives rise to stress tolerance, and in several models, HSP70 has been identified as being the main HSP responsible for resistance to future insults (Angelidis et al., 1991; Gabai et al., 1997; Gabai et al., 2000; Li et al., 1996; Mosser et al., 1997). The protective nature of HSP70 and others HSPs was originally attributed exclusively to their role as molecular chaperones that prevented stress-induced protein misfolding and aggregation, and that accelerated refolding (Young et al., 2004). However, recent findings have demonstrated that, in addition to this function, HSP70 suppresses apoptosis by directly inhibiting components of the JNK signaling pathway (Gabai et al., 2002; Gabai et al., 2000; Parcellier et al., 2003; Park et al., 2001; Yaglom et al., 1999). This inhibition involves direct binding of HSP70 to JNK (Park et al., 2001). The precise HSP70-JNK binding domains have not been identified, but available data suggest that HSP70 binds JNK at, or close to, the docking groove where interactions with both JNK targets and activators occur. It is thus likely that the HSP70-JNK association attenuates the interaction of JNK with upstream MKKs and/or downstream targets (Park et al., 2001). Inhibition of JNK by HSP70 does not appear to be directly related to its chaperone function since HSP70 mutants that lack chaperone function still inhibit JNK; furthermore, HSP70 can inhibit JNK activation even in the absence of stress-induced protein damage (Gabai et al., 2002; Park et al., 2001; Yaglom et al., 1999).

Our data show that compound 1 exposure results in the accumulation of HSP25 and HSP70 mRNA and protein in PC-12 cells. Transcriptional regulation of these HSPs normally requires the action of the HSF1 transcription factor. Transcriptional reporter assays demonstrated that compound 1 activates HSF1 transcriptional activity and comparison of wild-type MEFs with those lacking HSF1 established that compound 1 induces HSP25 and HSP70 production through an HSF1-dependent pathway. HSF1 is normally maintained in a latent form by virtue of its association with HSP90 (Zou et al., 1998). Association of compound 1 with HSP90 releases HSF1 and thereby facilitates HSP25 and HSP70 transcription. Consistent with this, we found that purified HSP90 directly binds compound 1 in pullout assays.

HSP90 is composed of three main domains. The C-terminal domain contains the HSP90 dimerization site as well as docking sites for various co-chaperones. The central domain contains a large hydrophobic surface that is involved in the binding of HSP90 client proteins and the N-terminal region contains the molecule's ATPase domain (reviewed in (Workman, 2004)). Unlike other chaperones, most known client proteins of HSP90 are involved in the regulation of survival and growth (reviewed in (Goetz et al., 2003)). Geldanamycin binds the ATP-binding pocket of HSP90 and this leads to allosteric changes in HSP90 that result in release of HSF1 and subsequent expression of HSP25 and HSP70 (Goetz et al., 2003; Sreedhar et al., 2004; Workman, 2004; Zou et al., 1998). Because this mechanism is similar to that which we propose for compound 1 and its analogs, it is reasonable to expect that the cellular effects of compound 1 and geldanamyin treatment will be similar. However, although scattered reports indicate that geldanamycin does confer protection to cells from protein damaging stress in vitro and in vivo, the majority of studies have shown that geldanamycin is cytotoxic and kills variety of normal and transformed cells, including PC12 cells (reviewed in (Goetz et al., 2003; Lopez-Maderuelo et al., 2001). The toxic effect of geldanamycin is due to the fact that geldanamycin not only releases bound HSF1 but, by occupying the ATP binding pocket, blocks HSP90 chaperone activity and reduces the stability and activity of HSP90 client proteins that include Akt and Raf (Fujita et al., 2002; Hostein et al., 2001; Kim et al., 2003; Nimmanapalli et al., 2001; Schulte et al., 1995; Schulte et al., 1996). There are major structural and mechanistic differences between geldanamycin and compound 1 and it appears unlikely that compound 1 binds to the N-terminal ATP binding domain of HSP90, as does geldanamycin. We have shown that compound 1 does not occupy the ATP-binding pocket of HSP90 and demonstrated that, unlike geldanamycin, compound 1 does not reduce levels of Akt or block its phosphorylation but may instead enhance Akt phosphorylation. An enhancement of AKT and Raf activity is observed during the misfolded protein response; misfolded proteins interact with the peptide-binding domains of HSP90 and cause the release of HSF-1 and our working hypothesis is that compound 1 interacts with a portion of the peptide-binding domain of HSP90 and this facilitates HSF-1 release while retaining HSP90 chaperone activity.

The HSP chaperone proteins serve a diverse set of roles. One of these includes the binding to and stabilizing the proper folding of a number of intercellular proteins, ensuring their activity, for example HSF1. Alternatively, HSP binding may inhibit the function of a protein, for example HSP90 binding to JNK. The HSPs have been shown to have mild ATP-ase activity, but the end result of this ATP-ase activity is not fully understood. Binding of typical HSP90 inhibitors, those which bind to the N-terminal ATP binding site, are believed to be active by causing conformational changes in the N-terminus of HSP90, thereby conferring altered chaperoning activity.

The imidazo[2,1-b-]1,3,4-thiadiazole-2-sulfonamides, imidazo[2,1-b-]1,3,4-thiadiazole-2-sulfones and imidazo[2,1-b-]1,3,4-thiadiazole-2-sulfoxides do not appear to bind to the N-terminal ATP binding site, as shown by two pieces of evidence, i) compound 1 effects HSF1 release from HSP90 while geldanymycin, the prototypical N-terminal ATP binding compound, does not, and ii) compound 1 augments the binding of HSP90 to ATP-sepharose beads.

In conclusion, we have identified a class of compounds that inhibit JNK activation by inducing production of HSP proteins. Several methods used to confirm the binding of compounds of formula I to HSP90 protein can be used to identify other compounds which bind to the same binding site as compounds of formula I. We anticipate that compounds which bind HSP90 in a similar manner to those of formula I, will serve as useful tools for basic research, and have therapeutic potential for the treatment of acute and chronic neurological disorders.

### Synthesis and Methodology

6-Arylimidazolo[2,1-b-]-1,3,4-thiadiazole-2-sulfonamide derivates may be prepared which are radiolabeled in a number of positions, using a variety of radioisotopes. The general synthesis of these 6-arylimidazolo[2,1-b-]-1,3,4-thiadiazole-2-sulfonamides can be carried out as illustrated below. Bromination of an appropriately substituted and radiolabeled acetophenone provides the corresponding 2-bromoacetophenone. Condensation of the these 2-bromoacetopheones with 2-amino-1,3,4-thiadiazole-5-sulfonamide in an appropriate solvent system provides the 6-arylimidazolo[2,1-b-]-1,3,4-thiadiazole-2-sulfonamide derivates which can be radiolabeled at the C5 position, on the aromatic ring, or as a substituent of the aromatic ring. Functional group changes may be incorporated at any point in the synthesis to incorporate radiolabels using methods known in the art.

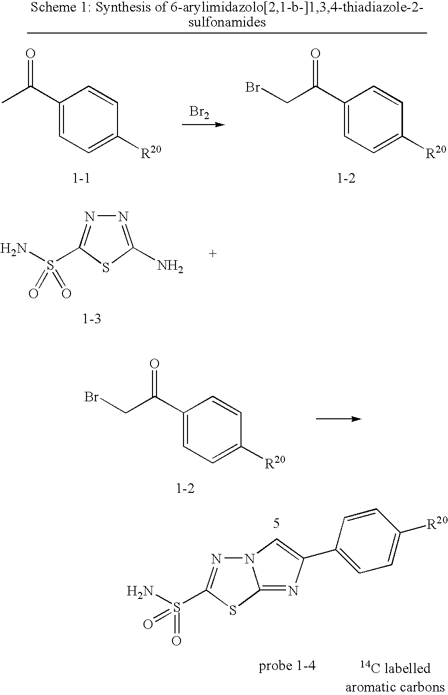

The following discussion illustrates several examples of radiolabeled acetophenones which can be used in the synthesis of 6-arylimidazolo[2,1-b-]1,3,4-thiadiazole-2-sulfonamide, according to Scheme 1. [¹⁴C]Acetophenone, either labeled at the acetyl or the aromatic carbons maybe used directly as in Scheme 1. Substituted acetophenone may be prepared by Friedel-Crafts acylation of bromobenzene or chlorobenzene, using [1-¹⁴C]acetic anhydride or [1-¹⁴C]acetyl chloride will provide [1-¹⁴C]acetophenones which can then be incorporated into the 6-arylimidazolo[2,1-b-]1,3,4-thiadiazole-2-sulfonamide synthesis illustrated in Scheme 1. Alternatively, [³H]acetic anhydride may be used in to provide [1,1,1-³H]acetophenones. Further functionalization of the acetophenone is possible, for example, 4′-bromoacetophenone my be functionalized using Suzuki couplings to provide 1-(biphenyl-4-yl)ethanones or Ullmann type reactions to provide 1-(4-phenoxyphenyl)ethanones.

The aromatic carbons of the acetophenone may be radiolabeled by acylating bromo[U-¹⁴C]benzene or chloro[U-¹⁴C]benzene with acetyl chloride, to provide [1′, 2′, 3′, 4′, 5′,6′-¹⁴C]acetophenones.

Alkylation of 4′-hydroxyacetophenone with either [¹⁴C]methyl iodide or [³H]methyl iodide will provide 4′-[¹⁴C]methoxyacetophenone and 4′-[³H]methoxyacetophenone, respectively.

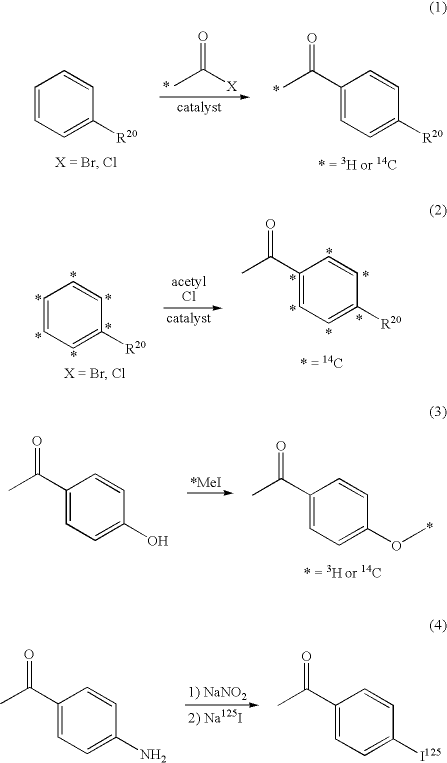

The reaction of 4′-aminoacetophenone with NaNO₂ and Na¹²⁵I provides 4′-{¹²⁵I]iodoacetophenone, which when further elaborated according to Scheme 1 will provide 6-([¹²⁵I]4-iodophenyl)imidazolo[2,1-b-]1,3,4-thiadiazole-2-sulfonamide.

Many combinations of the above methods may be envisioned. Representative examples of these compounds are listed below:

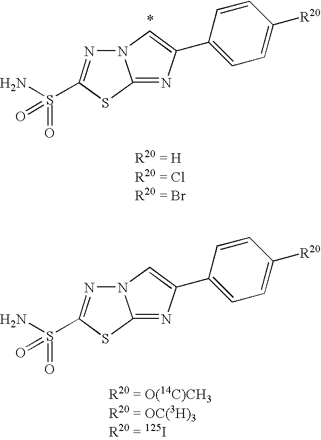

Appropriately substituted 6-arylimidazolo[2,1-b-]1,3,4-thiadiazole-2-sulfonamide derivates may be iodinated at the C5 carbon by treatment with iodine. The use if ¹²⁵I₂ therefore will provide 6-aryl-5-[¹²⁵I]iodoimidazolo[2,1-b]1,3,4-thiadiazole-2-sulfonamide derivates:

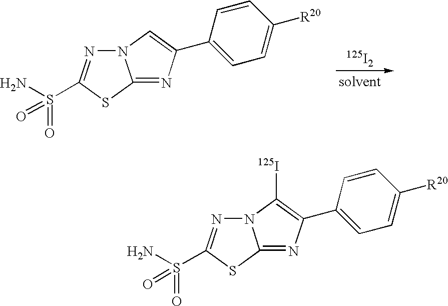

6-Arylimidazolo[2,1-b-]1,3,4-thiadiazole-2-sulfones and sulfoxides are also accessible to labeling. This class of compounds, represented by compounds in Scheme 2 have been used to pull down HSP90 and resemble the 6-arylimidazolo[2,1-b-]1,3,4-thiadiazole-2-sulfonamides in their biological activity.

Radioisotope labeling on either the right or left hand side of the molecule can be achieved: representation of this method is shown for the sulfoxides, in Scheme 2.

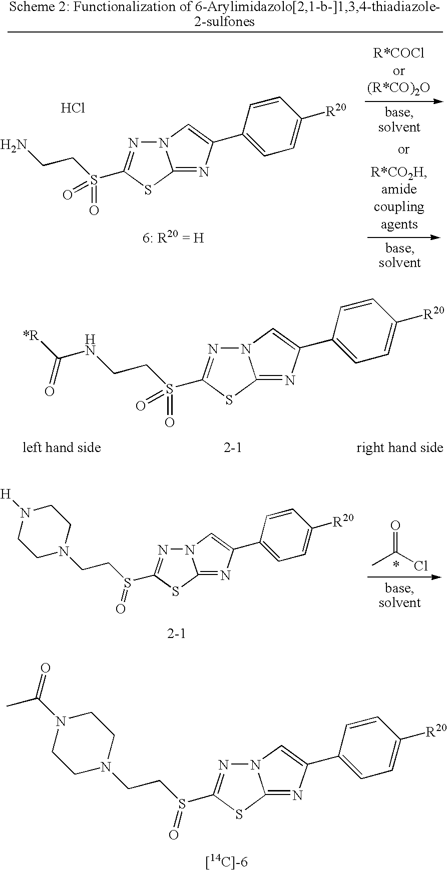

As such, the radiolabel may be incorporated in the right hand side of molecule, acyl group, or in the right hand side of the molecule, C5 of the imidazolo[2,1-b]-1,3,4-thiadiazole, aromatic C's, or aromatic substitution as described above.

Various radiolabeled acylating group may be used such as [1-¹⁴C]acetyl chloride, [2-¹⁴C]acetyl chloride, [1-¹⁴C]acetic anhydride, [³H]acetic anhydride, N-succinimidyl[2, 3-³H]propionate ([³H]NSP, or the Bolton & Hunter reagent for protein iodination, N-succinimidyl-3-(4-hydroxy-3-[¹²⁵I]iodophenyl)propionate

Activation of the carboxylic groups of the following reagents using amide coupling agents will also allow for acylation of the left hand side of the molecule; for example [carboxyl-¹⁴C]benzoic acid, [ring-U-¹⁴C]benzoic acid, and d-[8,9-³H]biotin.

Various radiolabeled amino acids may be appropriately protected at their side chains and coupled through their carboxylic acids to compound 3 using amide coupling agents. A selection of these radiolabeled amino acids include the following: [1-¹⁴C]glycine, [³H]glycine, 3-[5(n)-³H]indolylacetic acid, L-[4,5-³H]isoleucine, L-[4,5-³H]leucine, L-[4,5-³H]lysine monohydrochloride, L-[methyl-³H]methionine, 1-[4-³H]phenylalanine, I-[2,6-³H]phenylalanine, I-[U-¹⁴C]serine, I-[3-¹⁴C]serine, L-[U-¹⁴C]serine, I-[2,6-³H]phenylalanine, I-[4-³H]phenylalanine, and I-[U-¹⁴C]valine.

Alternatively, the sulfoxide compounds above may be alkylated with iodo[1-¹⁴C]acetamide to provide the following compounds:

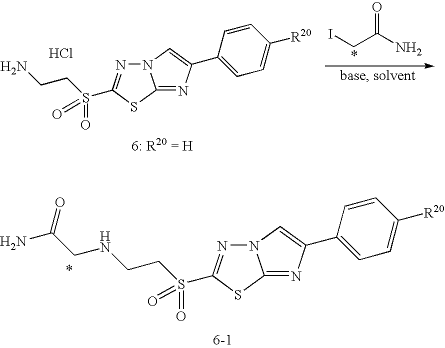

These approaches may be used to introduce fluorescent probes such as 4(5)-(Iodoacetamido)fluorescein, 5-carboxyfluorescein, coumarin based probes, Bodipy based probes, and others.

Also, these approaches may be used to introduce fluorescent probes such as 4(5)-(Iodoacetamido)fluorescein, 4(5)-carboxyfluorescein, coumarin based probes, BODIPY based probes, and others. Various commercial or readily available fluorescent reagents are available which display linker groups of various sizes and composition, which allow one to adjust the distance, flexibility and solubility parameters between the imidazo[2,1-b]-1,3,4-thiadiazole moiety and the fluorescent probe.

For example, treatment of the compound 6.TFA with 4(5)-(Iodoacetamido)fluorescein (1 equiv) and a base such as DIPEA (2 equiv), in a solvent such as THF will provide the corresponding fluorescein labeled compound 8:

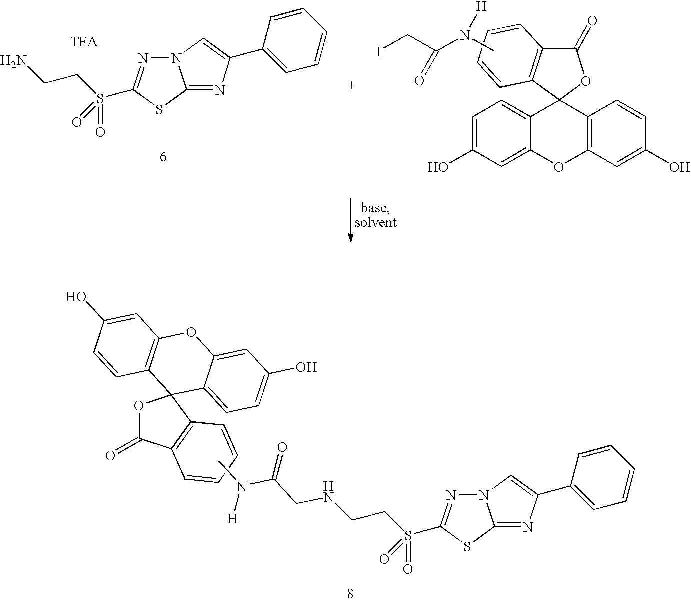

Alternatively, 4(5)-carboxyfluorescein N-succinimidyl ester (1 equiv) may be used to acylate compound 6 (1 equiv) in the presence of a base such as DIPEA (2 equiv) and a solvent such as THF, to provide the corresponding fluorescein labeled compound 9:

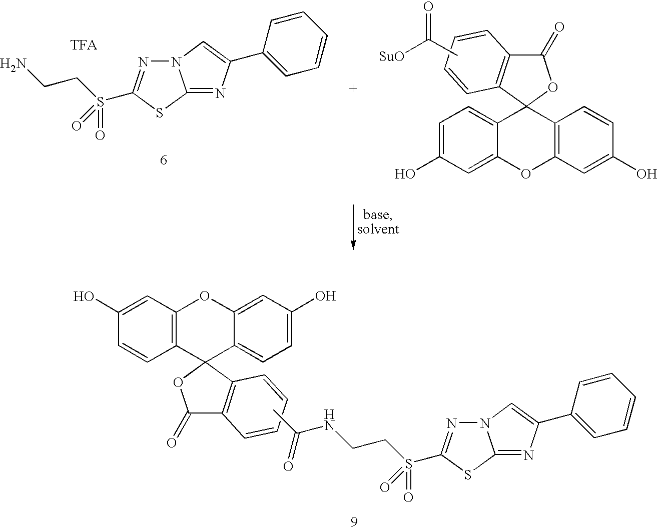

The carboxylic acid of 4(5)-carboxyfluorescein may alternatively be activated by the use of various amide coupling agents.

A similar method will yield a BODIPY labeled probe:

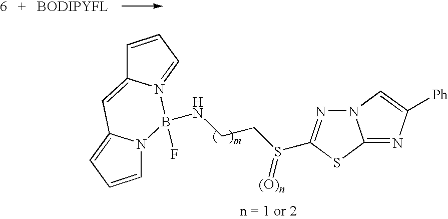

### Experimental Procedures

### Materials.

Compounds 1, 2, 3, 4, 5, and 12 to 19 were prepared according to procedures described in WO 03/051,890 A1. Compounds 6, 7, 10, 11 and 20 were prepared according to procedures described in WO 2004/111,060 A1.

| Compound | Structure |
|---|---|
| 1 | 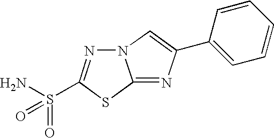 |
| 2 | 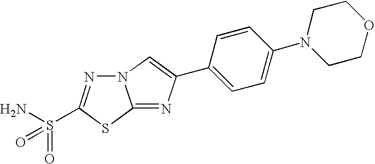 |
| 3 | 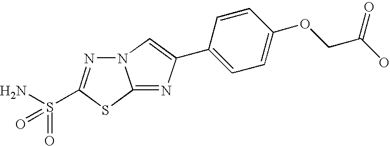 |
| 4 | 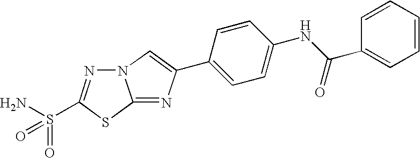 |
| 5 | 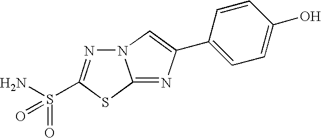 |
| 6 | 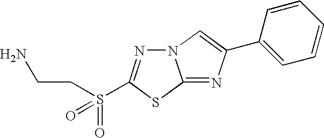 |
| 7 | 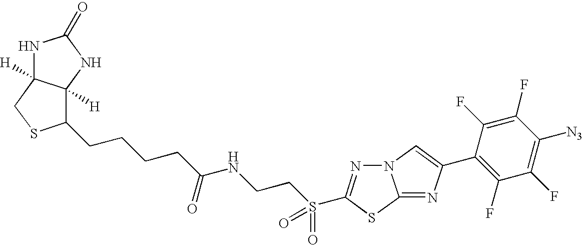 |
| 10 | 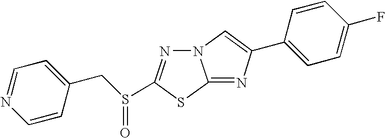 |
| 11 | 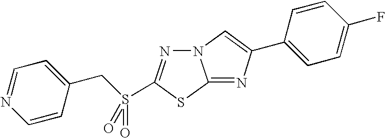 |
| 12 | 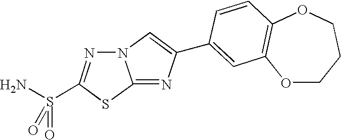 |
| 13 | 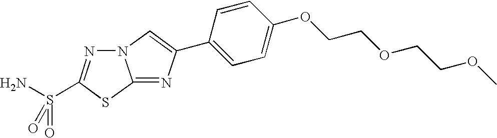 |
| 14 | 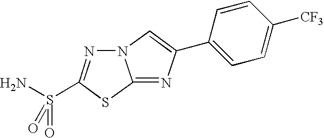 |
| 15 | 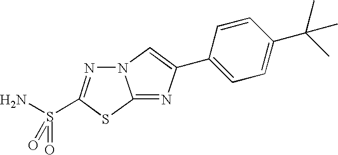 |
| 16 | 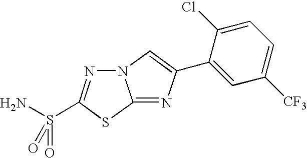 |
| 17 | 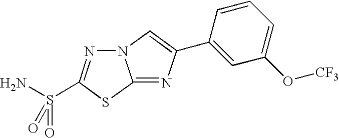 |
| 18 | 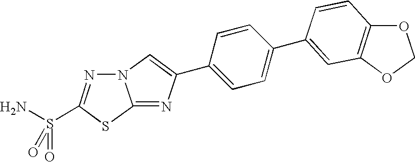 |
| 19 | 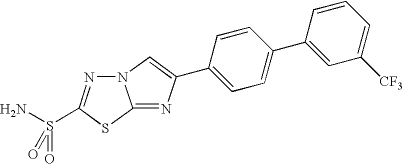 |
| 20 | 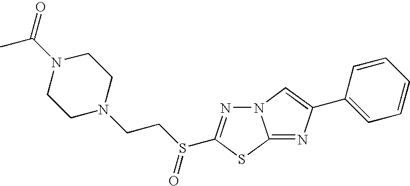 |

### 1. Synthesis of Probes

### A. Synthesis of [5-¹⁴C]-6-(3′-(trifluoromethyl)biphenyl-4-yl)imidazo[2,1-b]-1,3,4-thiadiazole-2-sulfonamide Mono Sodium Salt, [¹⁴C]—Na-probe 19

Compound [¹⁴C]—Na-probe 19 was prepared by the procedure described in Scheme 3 and 4. Scheme 3 illustrates the synthesis of the ¹⁴C radiolabeled acetophenone used in the preparation of [¹⁴C]-probe 19 and [¹⁴C]—Na-probe 19.

Suzuki coupling of 3-(trifluoromethyl)benzene boronic acid, 3-1, to ethyl 4-iodobenzoate, 3-2, provided the biphenyl ester 3-3. The radiolabel was introduced via treatment of 3-3 with the dilithio salt of [¹⁴C]methyl phenyl sulfone, 3-5, to provide sulfone 3-6. Reduction of the sulfone with Zn in ethanol provide [2-¹⁴C]-1-(3′-(trifluoromethyl)biphenyl-4-yl)ethanone, 3.8.

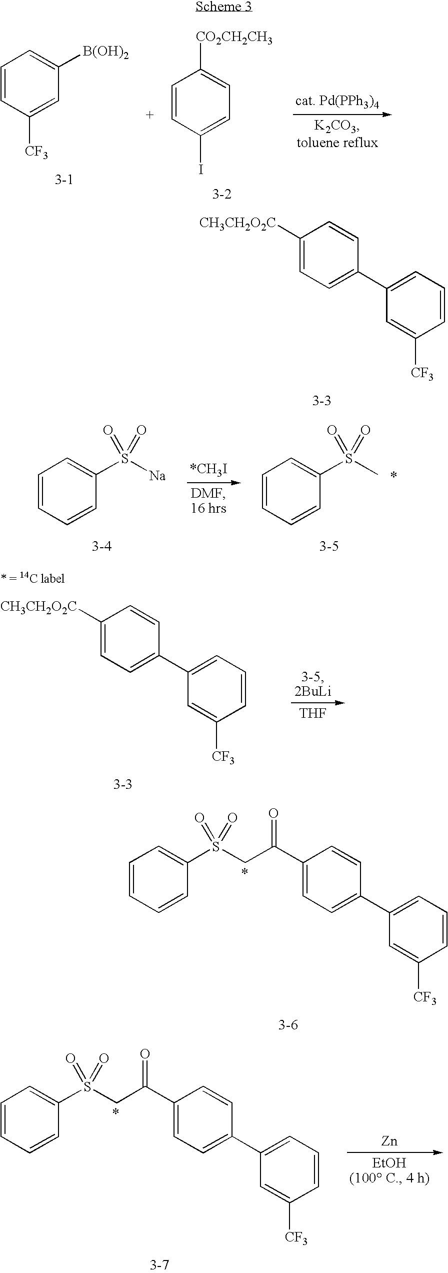

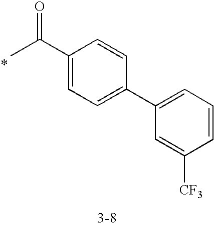

### B. Preparation of [2-¹⁴C]-1-(3′-(trifluoromethyl)biphenyl-4-yl)ethanone

Bromination of [2-¹⁴C]-1-(3′-(trifluoromethyl)biphenyl-4-yl)ethanone, 3-8, provided the bromo compound 4-1, which was condensed with 2-amino-1,3,4-thiadiazole-5-sulfonamide, 1-3, to provide [¹⁴C]-probe 19.

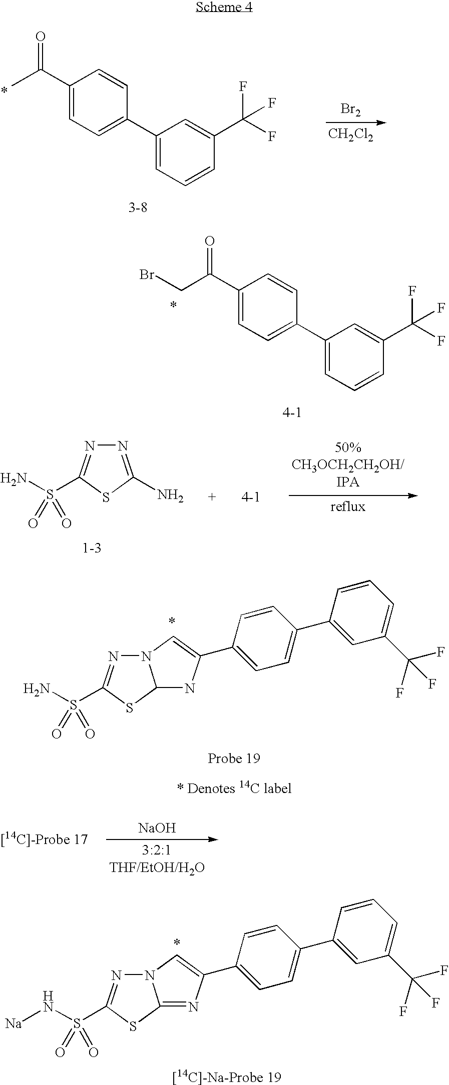

### C. Preparation of [¹⁴C]—Na-probe 19

Conversion of [¹⁴C]-probe 19 to its corresponding sodium salt was carried out using NaOH in a 3:2:1 THF/EthOH/water solution to provide [¹⁴C]—Na-probe 19 as a pail yellow solid

Purity: 99.62%

Specific activity: 22 uCi/mg

### D. Preparation of Fluorescein Labeled Probe

Compound 6 (90 mg, 0.2 mmol) and 4(5)-carboxyfluorescein N-succinimidyl ester (102 mg, 0.2 mmol) were dissolved in dry DMF (2 mL) and treated with DIPEA (38 μL, 0.22 mmol). The mixture was stirred for 18 hrs. Water was added slowly (10 mL) and the resulting precipitate was filtered off, washed with water and dried in vacuo. The resulting solid was purified by silica gel chromatography, eluting with THF, to provide an orange solid which was further purified by triturated with diethyl ether, providing 9 as an orange solid (70 mg, 52%). MS (m/z) M+1=667.0.

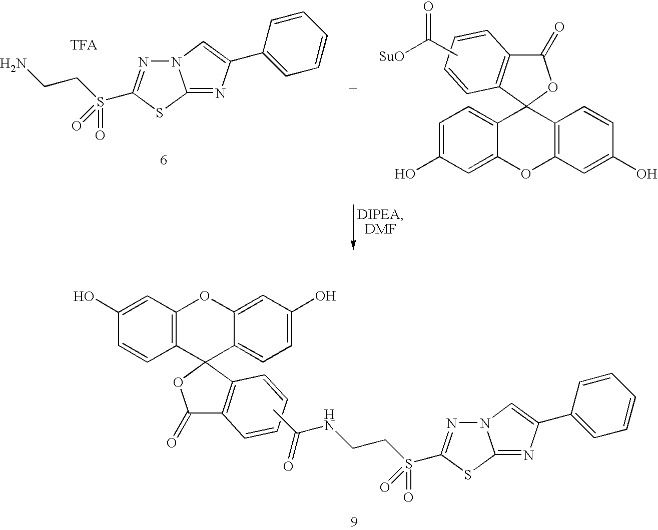

### E. Preparation of Compound 6-beads

Compound 6 was coupled to Affi-Gel 10 beads (BioRad) in an anhydrous reaction. 3 ml of Affi-Gel 10 beads were washed 3 times with 10 ml Isopropanol. 1.5 ml of DMSO was added to the washed beads. 750 uL of gel slurry was mixed with 500 ul of 20 mM compound 6 in DMSO. The reaction was incubated for 2 hours at room temperature. Remaining active sites on the gel were neutralized by adding 500 ul of 200 mM ethanolamine and incubating for a further 30 minutes at room temperature. Control beads were created by reacting the gel with ethanolamine alone. Conjugated beads were washed twice with 10 mL DMSO and then 3 times with PBS. The compound 6-beads were re-suspended as a 50% slurry in PBS and stored at 4° C.

### 2. Assays

Antibodies directed against JNK1 and IkBα were from Santa Cruz Biotechnology; those against C-Jun, phospho-c-Jun and cleaved caspase-3 were from Cell Signaling Technology; those against HSP25, HSP40, and HSP70 were from Stressgen; and that against actin was from ICN. Antibodies directed against NRAGE, and p75NTR have been previously described (Majdan et al., 1997; Salehi et al., 2002). The PC12^{rtTA} cell line was purchased from Clontech. HSP70 specific small interfering RNAs consisted of a ‘SmartPool’ mixture was purchased from Dharmacon. Recombinant adenovirus driving expression of β-galactosidase, p75NTR and NRAGE have been previously described (Roux et al., 2001))

Cell culture, infection, transfection and immunoblotting. Sympathetic neurons were maintained and deprived of NGF as previously described (Bamji et al., 1998; Ma et al., 1992). PC12^{rtTA} cells were maintained and infected as previously described (Majdan et al., 1997; Salehi et al., 2002); for NRAGE, 1 pg/ml doxycycline was added to all plates at time of infection. Wildtype and HSF1 nullizygous immortalized mouse embryonic fibroblasts (MEFs) were maintained in DMEM containing 10% fetal calf serum, 1 μM β-mercaptoethanol, 1% non-essential amino acids, 1% L-glutamine, 1% antimycotic solution and 1% sodium pyruvate (all from GibcoBRL). Sympathetic neuron survival was assessed using the MTS assay according to the manufacturer's instructions (Promega). Analysis of PC12 cell death was determined using a lactate dehydrogenase (LDH) assay (Roche) as per the manufacturer's instructions. Transfections using plasmids or RNAi were performed using Lipofectamine 2000. Immunoblotting was performed as previously described (Majdan et al., 1997; Salehi et al., 2002).

Detection of endogenous phosphorylation of GST-c-Jun. The cDNA region corresponding to amino acids 2-79 of human c-Jun was cloned into a mammalian GST expression vector (Mizushima and Nagata, 1990) and this was used to transfect PC12 cells. Lysates of transfected cells were prepared and incubated with 20 μl of glutathione-conjugated beads (Pharmacia) for 1 hour at 4° C. Beads were washed three times, resuspended in Lammeli sample buffer and incubated at 100° C. for 5 minutes. The level of GST-c-Jun phosphorylation was assessed by immunoblotting with a phospho-c-Jun specific antibody.

RT-PCR. PC12 cells were treated with increasing concentrations of compound 1 for 18 hours and mRNA was isolated using RNEasy Mini kits (Qiagen). cDNA was generated using the Omniscript RT kit (Qiagen) and random hexamers (Roche) as primers. PCR was performed using primer pairs directed against rat HSP70, HSP25 and actin (primer sequences and PCR conditions available upon request).

Transcriptional assays. MEFs were transfected with pGL3B-HSP70 or with the corresponding parental vector. compound 1 (40 μM) was added to the cells the next day and cells were harvested 48 hours after transfection. Transcriptional assays were performed using a luciferase assay system purchased from Promega.

ATP-Sepharose Interaction Assays. These were performed essentially as described in (Grenert et al., 1997). 1 ug of purified HSP90 protein was pre-incubated with ATP, geldanamycin or compound 1 in 200 ul incubation buffer (10 mM Tris pH 7.5, 50 mM KCl, 5 mM MgCl2, 2 mM DTT, 20 mM Na2MoO4, and 0.01% NP-40) for 10 min at RT. 25 ul of ATP-sepharose beads were added and the reactions were incubated for 30 min at 30° C. After thorough washing with ice-cold incubation buffer, bound proteins were eluted in sample buffer and HSP90 content was determined by immunoblotting.

Statistical analysis. For quantitation, each condition was performed in triplicate or quadruplicate, and results were analyzed by multiple analysis of variance with statistical probabilities assigned using the Tukey test for multiple comparisons.

### REFERENCES CITED

Adams, J. M. (2003). Ways of dying: multiple pathways to apoptosis. Genes Dev 17, 2481-2495.
Angel, P., Hattori, K., Smeal, T., and Karin, M. (1988). The jun proto-oncogene is positively autoregulated by its product, Jun/AP-1. Cell 55, 875-885.
Angelidis, C. E., Lazaridis, I., and Pagoulatos, G. N. (1991). Constitutive expression of heat-shock protein 70 in mammalian cells confers thermoresistance. Eur J Biochem 199, 35-39.
Bamji, S. X., Majdan, M., Pozniak, C. D., Belliveau, D. J., Aloyz, R., Kohn, J., Causing, C. G., and Miller, F. D. (1998). The p75 neurotrophin receptor mediates neuronal apoptosis and is essential for naturally occurring sympathetic neuron death. J Cell Biol 140, 911-923.
Bazenet, C. E., Mota, M. A., and Rubin, L. L. (1998). The small GTP-binding protein Cdc42 is required for nerve growth factor withdrawal-induced neuronal death. Proc Natl Acad Sci USA 95, 3984-3989.
Bennett, B. L., Sasaki, D. T., Murray, B. W., O'Leary, E. C., Sakata, S. T., Xu, W., Leisten, J. C., Motiwala, A., Pierce, S., Satoh, Y., et al. (2001). SP600125, an anthrapyrazolone inhibitor of Jun N-terminal kinase. Proc Natl Acad Sci USA 98, 13681-13686.
Bhakar, A. L., Howell, J. L., Paul, C. E., Salehi, A. H., Becker, E. B., Said, F., Bonni, A., and Barker, P. A. (2003). Apoptosis induced by p75NTR overexpression requires Jun kinase-dependent phosphorylation of Bad. J Neurosci 23, 11373-11381.
Davis, R. J. (2000). Signal transduction by the JNK group of MAP kinases. Cell 103, 239-252.
Dickens, M., Rogers, J. S., Cavanagh, J., Raitano, A., Xia, Z., Halpern, J. R., Greenberg, M. E., Sawyers, C. L., and Davis, R. J. (1997). A cytoplasmic inhibitor of the JNK signal transduction pathway. Science 277, 693-696.
Eilers, A., Whitfield, J., Babij, C., Rubin, L. L., and Ham, J. (1998). Role of the Jun kinase pathway in the regulation of c-Jun expression and apoptosis in sympathetic neurons. J Neurosci 18, 1713-1724.
Eilers, A., Whiffield, J., Shah, B., Spadoni, C., Desmond, H., and Ham, J. (2001). Direct inhibition of c-Jun N-terminal kinase in sympathetic neurones prevents c-jun promoter activation and NGF withdrawal-induced death. J Neurochem 76, 1439-1454.
Estus, S., Zaks, W. J., Freeman, R. S., Gruda, M., Bravo, R., and Johnson, E. M., Jr. (1994). Altered gene expression in neurons during programmed cell death: identification of c-jun as necessary for neuronal apoptosis. J Cell Biol 127, 1717-1727.
Figueroa-Masot, X. A., Hetman, M., Higgins, M. J., Kokot, N., and Xia, Z. (2001). Taxol induces apoptosis in cortical neurons by a mechanism independent of Bcl-2 phosphorylation. J Neurosci 21, 4657-4667.
Fujita, N., Sato, S., Ishida, A., and Tsuruo, T. (2002). Involvement of Hsp90 in signaling and stability of 3-phosphoinositide-dependent kinase-1. J Biol Chem 277, 10346-10353.
Gabai, V. L., Mabuchi, K., Mosser, D. D., and Sherman, M. Y. (2002). Hsp72 and stress kinase c-jun N-terminal kinase regulate the bid-dependent pathway in tumor necrosis factor-induced apoptosis. Mol Cell Biol 22, 3415-3424.
Gabai, V. L., Meriin, A. B., Mosser, D. D., Caron, A. W., Rits, S., Shifrin, V. I., and Sherman, M. Y. (1997). Hsp70 prevents activation of stress kinases. A novel pathway of cellular thermotolerance. J Biol Chem 272, 18033-18037.
Gabai, V. L., Yaglom, J. A., Volloch, V., Meriin, A. B., Force, T., Koutroumanis, M., Massie, B., Mosser, D. D., and Sherman, M. Y. (2000). Hsp72-mediated suppression of c-Jun N-terminal kinase is implicated in development of tolerance to caspase-independent cell death. Mol Cell Biol 20, 6826-6836.
Goetz, M. P., Toft, D. O., Ames, M. M., and Erlichman, C. (2003). The Hsp90 chaperone complex as a novel target for cancer therapy. Ann Oncol 14, 1169-1176.
Grenert, J. P., Sullivan, W. P., Fadden, P., Haystead, T. A., Clark, J., Mimnaugh, E., Krutzsch, H., Ochel, H. J., Schulte, T. W., Sausville, E., et al. (1997). The amino-terminal domain of heat shock protein 90 (hsp90) that binds geldanamycin is an ATP/ADP switch domain that regulates hsp90 conformation. J Biol Chem 272, 23843-23850.
Hujduk, P J. (2006) SAR by NMR: putting the pieces together. Mol. Interv. 2006, 6, 266-72.
Ham, J., Babij, C., Whitfield, J., Pfarr, C. M., Lallemand, D., Yaniv, M., and Rubin, L. L. (1995). A c-Jun dominant negative mutant protects sympathetic neurons against programmed cell death. Neuron 14, 927-939.
Harding, T. C., Xue, L., Bienemann, A., Haywood, D., Dickens, M., Tolkovsky, A. M., and Uney, J. B. (2001). Inhibition of JNK by overexpression of the JNL binding domain of JIP-1 prevents apoptosis in sympathetic neurons. J Biol Chem 276, 4531-4534.
Harper, S. J., and LoGrasso, P. (2001). Signalling for survival and death in neurones: the role of stress-activated kinases, JNK and p38. Cell Signal 13, 299-310.
Harris, C. A., and Johnson, E. M., Jr. (2001). BH3-only Bcl-2 family members are coordinately regulated by the JNK pathway and require Bax to induce apoptosis in neurons. J Biol Chem 276, 37754-37760.
Hostein, I., Robertson, D., DiStefano, F., Workman, P., and Clarke, P. A. (2001). Inhibition of signal transduction by the Hsp90 inhibitor 17-allylamino-17-demethoxygeldanamycin results in cytostasis and apoptosis. Cancer Res 61, 4003-4009.
Kim, S., Kang, J., Hu, W., Evers, B. M., and Chung, D. H. (2003). Geldanamycin decreases Raf-1 and Akt levels and induces apoptosis in neuroblastomas. Int J Cancer 103, 352-359.
Lee, L. F., Li, G., Templeton, D. J., and Ting, J. P. (1998). Paclitaxel (Taxolyinduced gene expression and cell death are both mediated by the activation of c-Jun NH2-terminal kinase (JNK/SAPK). J Biol Chem 273, 28253-28260.
Li, W. X., Chen, C. H., Ling, C. C., and Li, G. C. (1996). Apoptosis in heat-induced cell killing: the protective role of hsp-70 and the sensitization effect of the c-myc gene. Radiat Res 145, 324-330.
Lopez-Maderuelo, M. D., Fernandez-Renart, M., Moratilla, C., and Renart, J. (2001). Opposite effects of the Hsp90 inhibitor Geldanamycin: induction of apoptosis in PC12, and differentiation in N2A cells. FEBS Lett 490, 23-27.
Ma, Y., Campenot, R. B., and Miller, F. D. (1992). Concentration-dependent regulation of neuronal gene expression by nerve growth factor. J Cell Biol 117, 135-141.
Majdan, M., Lachance, C., Gloster, A., Aloyz, R., Zeindler, C., Bamji, S., Bhakar, A., Belliveau, D., Fawcett, J., Miller, F. D., and Barker, P. A. (1997). Transgenic mice expressing the intracellular domain of the p75 neurotrophin receptor undergo neuronal apoptosis. J Neurosci 17, 6988-6998.
Maroney, A. C., Finn, J. P., Connors, T. J., Durkin, J. T., Angeles, T., Gessner, G., Xu, Z., Meyer, S. L., Savage, M. J., Greene, L. A., et al. (2001). Cep-1347 (KT7515), a semisynthetic inhibitor of the mixed lineage kinase family. J Biol Chem 276, 25302-25308.
McMillan, D. R., Xiao, X., Shao, L., Graves, K., and Benjamin, I. J. (1998). Targeted disruption of heat shock transcription factor 1 abolishes thermotolerance and protection against heat-inducible apoptosis. J Biol Chem 273, 7523-7528
Mizushima, S., and Nagata, S. (1990). pEF-BOS, a powerful mammalian expression vector. Nucleic Acids Res 18, 5322.
Monaco, R., Friedman, F. K., Hyde, M. J., Chen, J. M., Manolatus, S., Adler, V., Ronai, Z., Koslosky, W., and Pincus, M. R. (1999). Identification of a glutathione-5-transferase effector domain for inhibition of jun kinase, by molecular dynamics. J Protein Chem 18, 859-866.
Mosser, D. D., Caron, A. W., Bourget, L., Denis-Larose, C., and Massie, B. (1997). Role of the human heat shock protein hsp70 in protection against stress-induced apoptosis. Mol Cell Biol 17, 5317-5327.
Muda, M., Theodosiou, A., Rodrigues, N., Boschert, U., Camps, M., Gillieron, C., Davies, K., Ashworth, A., and Arkinstall, S. (1996). The dual specificity phosphatases M36 and MKP-3 are highly selective for inactivation of distinct mitogen-activated protein kinases. J Biol Chem 271, 27205-27208.
Nimmanapalli, R., O'Bryan, E., and Bhalla, K. (2001). Geldanamycin and its analogue 17-allylamino-17-demethoxygeldanamycin lowers Bcr-Abl levels and induces apoptosis and differentiation of Bcr-Abl-positive human leukemic blasts. Cancer Res 61, 1799-1804.
Parcellier, A., Gurbuxani, S., Schmitt, E., Solary, E., and Gamido., C. (2003). Heat shock proteins, cellular chaperones that modulate mitochondrial cell death pathways. Biochem Biophys Res Commun 304, 505-512.
Park, H. S., Lee, J. S., Huh, S. H., Seo, J. S., and Choi, E. J. (2001). Hsp72 functions as a natural inhibitory protein of c-Jun N-terminal kinase. Embo J 20, 446-456.
Putcha, G. V., Moulder, K. L., Golden, J. P., Bouillet, P., Adams, J. A., Strasser, A., and Johnson, E. M. (2001). Induction of BIM, a proapoptotic BH3-only BCL-2 family member, is critical for neuronal apoptosis. Neuron 29, 615-628.
Roux, P. P., and Barker, P. A. (2002). Neurotrophin signaling through the p75 neurotrophin receptor. Prog Neurobiol 67, 203-233.
Roux, P. P., Bhakar, A. L., Kennedy, T. E., and Barker, P. A. (2001). The p75 neurotrophin receptor activates Akt (protein kinase B) through a phosphatidylinositol 3-kinase-dependent pathway. J Biol Chem 276, 23097-23104.
Salehi, A. H., Roux, P. P., Kubu, C. J., Zeindler, C., Bhakar, A., Tannis, L. L., Verdi, J. M., and Barker, P. A. (2000). NRAGE, a novel MAGE protein, interacts with the p75 neurotrophin receptor and facilitates nerve growth factor-dependent apoptosis. Neuron 27, 279-288.
Salehi, A. H., Xanthoudakis, S., and Barker, P. A. (2002). NRAGE, a p75 neurotrophin receptor-interacting protein, induces caspase activation and cell death through a JNK-dependent mitochondrial pathway. J Biol Chem 277, 48043-48050.
Sanchez-Perez, I., and Perona, R. (1999). Lack of c-Jun activity increases survival to cisplatin. FEBS Lett 453, 151-158.
Saporito, M. S., Hudkins, R. L., and Maroney, A. C. (2002). Discovery of CEP-1347/KT-7515, an inhibitor of the JNK/SAPK pathway for the treatment of neurodegenerative diseases. Prog Med Chem 40, 23-62.
Schulte, T. W., Blagosklonny, M. V., Ingui, C., and Neckers, L. (1995). Disruption of the Raf-1-Hsp90 molecular complex results in destabilization of Raf-1 and loss of Raf-1-Ras association. J Biol Chem 270, 24585-24588.
Schulte, T. W., Blagosklonny, M. V., Romanova, L., Mushinski, J. F., Monia, B. P., Johnston, J. F., Nguyen, P., Trepel, J., and Neckers, L. M. (1996). Destabilization of Raf-1 by geldanamycin leads to disruption of the Raf-1-MEK-mitogen-activated protein kinase signalling pathway. Mol Cell Biol 16, 5839-5845.
Shim, J., Lee, H., Park, J., Kim, H., and Choi, E. J. (1996). A non-enzymatic p21 protein inhibitor of stress-activated protein kinases. Nature 381, 804-806.
Shim, J., Park, H. S., Kim, M. J., Park, J., Park, E., Cho, S. G., Eom, S. J., Lee, H. W., Joe, C. O., and Choi, E. J. (2000). Rb protein down-regulates the stress-activated signals through inhibiting c-Jun N-terminal kinase/stress-activated protein kinase. J Biol Chem 275, 14107-14111.
Sreedhar, A. S., and Csermely, P. (2004). Heat shock proteins in the regulation of apoptosis: new strategies in tumor therapy: a comprehensive review. Pharmacol Ther 101, 227-257.
Sreedhar, A. S., Soti, C., and Csermely, P. (2004). Inhibition of Hsp90: a new strategy for inhibiting protein kinases. Biochim Biophys Acta 1697, 233-242.
Srivastava, R. K., Mi, Q. S., Hardwick, J. M., and Longo, D. L. (1999). Deletion of the loop region of Bcl-2 completely blocks paclitaxel-induced apoptosis. Proc Natl Acad Sci USA 96, 3775-3780.
Wang, W., Ma, C., Mao, Z., and Li, M. (2004). JNK inhibition as a potential strategy in treating Parkinson's disease. Drug News Perspect 17, 646-654.
Whiffield, J., Neame, S. J., Paquet, L., Bernard, O., and Ham, J. (2001). Dominant-negative c-Jun promotes neuronal survival by reducing BIM expression and inhibiting mitochondrial cytochrome c release. Neuron 29, 629-643.
Workman, P. (2004). Combinatorial attack on multistep oncogenesis by inhibiting the Hsp90 molecular chaperone. Cancer Lett 206, 149-157.
Yaglom, J. A., Gabai, V. L., Meriin, A. B., Mosser, D. D., and Sherman, M. Y. (1999). The function of HSP72 in suppression of c-Jun N-terminal kinase activation can be dissociated from its role in prevention of protein damage. J Biol Chem 274, 20223-20228.
Young, J. C., Agashe, V. R., Siegers, K., and Hartl, F. U. (2004). Pathways of chaperone-mediated protein folding in the cytosol. Nat Rev Mol Cell Biol 5, 781-791.
Zou, J., Guo, Y., Guettouche, T., Smith, D. F., and Voellmy, R. (1998). Repression of heat shock transcription factor HSF1 activation by HSP90 (HSP90 complex) that forms a stress-sensitive complex with HSF1. Cell 94, 471-480.

### Other Embodiments

From the foregoing description, it will be apparent to one of ordinary skill in the art that variations and modifications may be made to the invention described herein to adapt it to various usages and conditions. Such embodiments are also within the scope of the present invention.

All publications mentioned in this specification are hereby incorporated by reference.

All literature, patents, published patent applications cited herein are hereby incorporated by reference.

While specific embodiments have been described, those skilled in the art will recognize many alterations that could be made within the spirit of the invention, which is defined solely according to the following claims:

1. An assay for identifying compounds that modulate a neuronal apoptotic pathway, the assay comprising:
a) contacting HSP90 protein with a probe to form a probe:HSP90 complex, the probe being displaceable by a test compound;
b) measuring a signal from the probe so as to establish a reference level;
c) incubating the probe:HSP90 complex with the test compound;
d) measuring the signal from the probe;
e) comparing the signal from step d) with the reference level, a modulation of the signal indicating that the test compound binds to HSP90,
wherein the probe is a compound of Formula 1, and wherein the probe of Formula 1 is labeled with a detectable label and/or an affinity tag.

2. The assay, according to claim 1, in which the detectable label is selected from the group consisting of: a radioactive atom, a fluorescent label, a colorimetric label, and a chemiluminescent label.

3. The assay, according to claim 2, in which the detectable label is a radioactive atom.

4. The assay, according to claim 3, in which the radioactive atom is selected from the group consisting of: ³H, ¹⁴C, and ¹²⁵I.

5. The assay, according to claim 1, in which the detectable label is a fluorescent label.

6. The assay, according to claim 5, in which the fluorescent label is fluoroscene based, coumarin based, or Bodipy based.

7. The assay, according to claim 1, in which the probe comprises compounds of Formula I: 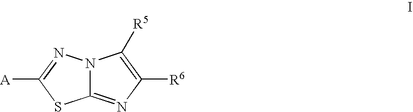
or a salt thereof,
wherein:
A is
1) —S(O)₂NR¹R²; or
2) —S(O)ₙR³;
n is 1 or 2;
m is 1 to 20;
Y is NH, O or S;
R¹ and R² are independently selected from:
1) H, or
2) C₁-C₆ alkyl;
R³ is:
1) C₁-C₆ alkyl,
2) haloalkyl,
3) aryl, or
4) heteroaryl,
wherein the alkyl is optionally substituted with one or more R¹⁵ substituents; and the aryl and heteroaryl are optionally substituted with one or more R²⁰ substituents;
R⁵ is:
1) H,
2) halogen,
3) C₁-C₆ alkyl, or
4) aryl;
R⁶ is
1) haloalkyl,
2) adamantyl,
3) aryl, or
4) heteroaryl,
wherein the aryl and the heteroaryl are optionally substituted with one or more substituents independently selected from R²⁰;
R⁷ is
1) H,
2) haloalkyl,
3) C₁-C₆ alkyl,
4) aryl,
5) heteroaryl or
6) heterocyclyl,
wherein the alkyl is optionally substituted with one or more R¹⁵ substituents; and wherein the aryl, heteroaryl and heterocyclyl is optionally substituted with one or more R²⁰ substituents;
R¹⁰ is
1) C₁-C₆ alkyl,
2) C₃-C₇ cycloalkyl,
3) haloalkyl,
4) C₂-C₆ alkenyl,
5) C₂-C₆ alkynyl,
6) C₅-C₇ cycloalkenyl,
7) aryl,
8) heteroaryl, or
9) heterocyclyl,
wherein the alkyl, cycloalkyl, alkenyl, alkynyl, cycloalkenyl are optionally substituted with one or more R¹⁵ substituents, and the aryl, heteroaryl, and heterocyclyl are optionally substituted with one or more R²⁰ substituents;
R¹¹ and R¹² are independently selected from:
1) H,
2) C₁-C₆ alkyl,
3) C₃-C₇ cycloalkyl,
4) haloalkyl,
5) aryl,
6) heteroaryl,
7) heterocyclyl,
8) C(O)—C₁-C₆ alkyl
9) C(O)—C₃-C₇ cycloalkyl
10) C(O)-aryl,
11) C(O)-heteroaryl,
12) C(O)-heterocyclyl,
13) C(O)Y—C₁-C₆ alkyl
14) C(O)Y—C₃-C₇ cycloalkyl
15) C(O)Y-aryl,
16) C(O)Y-heteroaryl,
17) C(O)Y-heterocyclyl,
wherein the alkyl and the cycloalkyl are optionally substituted with one or more R¹⁵ substituents, and the aryl, heteroaryl, and heterocyclyl are optionally substituted with one or more R²⁰ substituents;
or R¹¹ and R¹² together with the nitrogen atom to which they are bonded form a five, six or seven membered heterocyclic ring optionally substituted with one or more R²⁰ substituents;
R¹⁴ is
1) H, or
2) C₁-C₆ alkyl;
R¹⁵ is
1) NO₂,
2) CN,
3) halogen,
4) C₃-C₇ cycloalkyl,
5) haloalkyl,
6) aryl,
7) heteroaryl,
8) heterocyclyl,
9) OR⁷,
10) S(O)ₙR¹⁰,
11) NR¹¹R¹²,
12) CHO,
13) C(O)R¹⁰,
14) CO₂R¹⁴,
15) CONR¹¹R¹², or
16) S(O)ₙNR¹¹R¹²,
wherein the aryl and heteroaryl are optionally substituted with one or more R²⁵ substituents;
R²⁰ is
1) NO₂,
2) CN,
3) N₃,
4) (OR⁴)₂,
5) adamantyl,
6) halogen,
7) C₁-C₆ alkyl,
8) C₂-C₆ alkenyl,
9) C₂-C₄ alkynyl,
10) C₃-C₇ cycloalkyl,
11) C₃-C₇ cycloalkenyl,
12) aryl,
13) heteroaryl,
14) heterocyclyl,
15) haloalkyl,
16) OR⁷,
17) SR⁷,
18) S(O)ₙR¹⁰,
19) NR¹¹R¹²,
20) CHO,
21) C(O)R¹⁰,
22) C(O)OR¹⁴,
23) CONR¹¹R¹²,
24) S(O)ₙNR¹¹R¹²,
25) NR¹⁴C(O)R¹⁰,
26) NR¹⁴S(O)₂R¹⁰,
27) OC(O)R¹⁰,
28) SC(O)R¹⁰,
29) —O—(C₁-C₆alkyl-O)ₘR¹⁴, or
30) —O—(C₁-C₆ alkyl-O)ₘ—C₁-C₆ alkyl-OR¹⁴,
wherein the alkyl and the cycloalkyl are optionally substituted with one or more R¹⁵ substituents;
and wherein the aryl, heteroaryl and the heterocyclyl are optionally substituted with one or more R²⁵ substituents;
R²⁵ is
1) halogen,
2) NO₂,
3) CN,
4) B(OR¹⁴)₂,
5) haloalkyl,
6) C₁-C₆ alkyl,
7) C₂-C₆ alkenyl,
8) C₂-C₄ alkynyl,
9) OR⁷,
10) SR⁷
11) NR¹¹R¹²,
12) CHO,
13) C(O)R¹⁰,
14) C(O)OR¹⁴,
15) CONR¹¹R¹²,
16) S(O)₂R¹⁰,
17) S(O)₂NR¹¹R¹²,
18) NR¹⁴COR¹⁰,
19) NR¹⁴S(O)₂R¹⁰,
20) OC(O)R¹⁰, or
21) SC(O)R¹⁰;
wherein the probe is labeled with a detectable label or an affinity tag and wherein the probe binds to HSP90 protein.

8. The assay, according to claim 7, in which the probe is of the following formula: 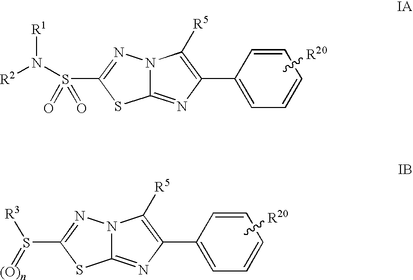
or a salt thereof,
wherein:
n is 1 or 2;
m is 1 to 20;
Y is NH, O or S;
R¹ and R² are independently selected from:
1) H, or
2) C₁-C₆ alkyl;
R³ is:
1) C₁-C₆ alkyl,
2) haloalkyl,
3) aryl, or
4) heteroaryl,
wherein the alkyl is optionally substituted with one or more R¹⁵ substituents; and the aryl and heteroaryl are optionally substituted with one or more R²⁰ substituents;
R⁵ is
1) H, or
2) halogen;
R⁷ is
1) H,
2) haloalkyl,
3) C₁-C₆ alkyl,
4) aryl,
5) heteroaryl or
6) heterocyclyl,
wherein the alkyl is optionally substituted with one or more R¹⁵ substituents; and wherein the aryl, heteroaryl and heterocyclyl is optionally substituted with one or more R²⁰ substituents;
R¹⁰ is
1) C₁-C₆ alkyl,
2) C₃-C₇ cycloalkyl,
3) haloalkyl,
4) C₂-C₆ alkenyl,
5) C₂-C₆ alkynyl,
6) C₅-C₇ cycloalkenyl,
7) aryl,
8) heteroaryl, or
9) heterocyclyl,
wherein the alkyl, cycloalkyl, alkenyl, alkynyl, cycloalkenyl are optionally substituted with one or more R¹⁵ substituents, and the aryl, heteroaryl, and heterocyclyl are optionally substituted with one or more R²⁰ substituents;
R¹¹ and R¹² are independently selected from:
1) H,
2) C₁-C₆ alkyl,
3) C₃-C₇ cycloalkyl,
4) haloalkyl,
5) aryl,
6) heteroaryl,
7) heterocyclyl,
8) C(O)—C₁-C₆ alkyl,
9) C(O)—C₃-C₇ cycloalkyl
10) C(O)-aryl,
11) C(O)-heteroaryl,
12) C(O)-heterocyclyl,
13) C(O)Y—C₁-C₆ alkyl
14) C(O)Y—C₃-C₇ cycloalkyl
15) C(O)Y-aryl,
16) C(O)Y-heteroaryl, or
17) C(O)Y-heterocyclyl,
wherein the alkyl and the cycloalkyl are optionally substituted with one or more R¹⁵ substituents, and the aryl, heteroaryl, and heterocyclyl are optionally substituted with one or more R²⁰ substituents;
or R¹¹ and R¹² together with the nitrogen atom to which they are bonded form a five, six or seven membered heterocyclic ring optionally substituted with one or more R²⁰ substituents;
R¹⁴ is
1) H, or
2) C₁-C₆ alkyl;
R¹⁵ is
1) NO₂,
2) CN,
3) halogen,
4) C₃-C₇ cycloalkyl,
5) haloalkyl,
6) aryl,
7) heteroaryl,
8) heterocyclyl,
9) OR⁷,
10) S(O)ₙR¹⁰,
11) NR¹¹R¹²,
12) CHO,
13) C(O)R¹⁰,
14) CO₂R¹⁴,
15) CONR¹¹R¹², or
16) S(O)ₙNR¹¹R¹²,
wherein the aryl and heteroaryl are optionally substituted with one or more R²⁵ substituents;
R²⁰ is
1) NO₂,
2) CN,
3) N₃,
4) B(OR¹⁴)₂,
5) adamantyl,
6) halogen,
7) C₁-C₆ alkyl,
8) C₂-C₆ alkenyl,
9) C₂-C₄ alkynyl,
10) C₃-C₇ cycloalkyl,
11) C₃-C₇ cycloalkenyl,
12) aryl,
13) heteroaryl,
14) heterocyclyl,
15) haloalkyl,
16) OR⁷,
17) SR⁷,
18) S(O)ₙR¹⁰,
19) NR¹¹R¹²,
20) CHO,
21) C(O)R¹⁰,
22) C(O)OR¹⁴,
23) CONR¹¹R¹²,
24) S(O)ₙNR¹¹R¹²,
25) NR¹⁴C(O)R¹⁰,
26) NR¹⁴S(O)₂R¹⁰,
27) OC(O)R¹⁰,
28) SC(O)R¹⁰,
29) —O—(C₁-C₆alkyl-O)ₘR¹⁴, or
30) —O—(C₁-C₆ alkyl-O)ₘ—C₁-C₆ alkyl-OR¹⁴,
wherein the alkyl and the cycloalkyl are optionally substituted with one or more R¹⁵ substituents;
and wherein the aryl, heteroaryl and the heterocyclyl are optionally substituted with one or more R²⁵ substituents;
R²⁵ is
1) halogen,
2) NO₂,
3) CN,
4) B(OR¹⁴)₂,
5) haloalkyl,
6) C₁-C₆ alkyl,
7) C₂-C₆ alkenyl,
8) C₂-C₄ alkynyl,
9) OR⁷,
10) SR⁷,
11) NR¹¹R¹²,
12) CHO,
13) C(O)R¹⁰,
14) C(O)OR¹⁴,
15) CONR¹¹R¹²,
16) S(O)₂R¹⁰,
17) S(O)₂NR¹¹R¹²,
18) NR¹⁴COR¹⁰,
19) NR¹⁴S(O)₂R¹⁰,
20) OC(O)R¹⁰, or
21) SC(O)R¹⁰;
wherein the probe comprises a detectable label or an affinity tag attached to any suitable position, and wherein the probe binds to HSP90 protein and is capable of being displaced by an inhibitor apoptotic JNK signaling pathway.

9. The assay, according to claim 8, in which the probe is of the following formula: 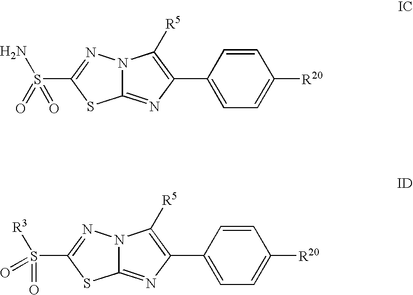
or a salt thereof,
wherein:
n is 1 or 2;
m is 1 to 20;
Y is NH, O or S;
R³ is C₁-C₆ alkyl optionally substituted with one or more R¹⁵ substituents;
R⁵ is
1) H, or
2) halogen;
R⁷ is
1) H,
2) haloalkyl,
3) C₁-C₆ alkyl,
4) aryl,
5) heteroaryl or
6) heterocyclyl,
wherein the alkyl is optionally substituted with one or more R¹⁵ substituents; and wherein the aryl, heteroaryl and heterocyclyl is optionally substituted with one or more R²⁰ substituents;
R¹⁰ is
1) C₁-C₆ alkyl,
2) C₃-C₇ cycloalkyl,
3) haloalkyl,
4) C₂-C₆ alkenyl,
5) C₂-C₆ alkynyl,
6) C₅-C₇ cycloalkenyl,
7) aryl,
8) heteroaryl, or
9) heterocyclyl,
wherein the alkyl, cycloalkyl, alkenyl, alkynyl, cycloalkenyl are optionally substituted with one or more R¹⁵ substituents, and the aryl, heteroaryl, and heterocyclyl are optionally substituted with one or more R²⁰ substituents;
R¹¹ and R¹² are independently selected from:
1) H,
2) C₁-C₆ alkyl,
3) C₃-C₇ cycloalkyl,
4) haloalkyl,
5) aryl,
6) heteroaryl,
7) heterocyclyl,
8) C(O)—C₁-C₆ alkyl,
9) C(O)—C₃-C₇ cycloalkyl
10) C(O)-aryl,
11) C(O)-heteroaryl,
12) C(O)-heterocyclyl,
13) C(O)Y—C₁-C₆ alkyl
14) C(O)Y—C₃-C₇ cycloalkyl
15) C(O)Y-aryl,
16) C(O)Y-heteroaryl, or
17) C(O)Y-heterocyclyl,
wherein the alkyl and the cycloalkyl are optionally substituted with one or more R¹⁵ substituents, and the aryl, heteroaryl, and heterocyclyl are optionally substituted with one or more R²⁰ substituents;
R¹⁴ is
1) H, or
2) C₁-C₆ alkyl;
R¹⁵ is
1) NO₂,
2) CN,
3) halogen,
4) C₃-C₇ cycloalkyl,
5) haloalkyl,
6) aryl,
7) heteroaryl,
8) heterocyclyl,
9) OR⁷,
10) S(O)ₙR¹⁰,
11) NR¹¹R¹²,
12) CHO,
13) C(O)R¹⁰,
14) CO₂R¹⁴
15) CONR¹¹R¹², or
16) S(O)ₙNR¹¹R¹²,
wherein the aryl and heteroaryl are optionally substituted with one or more R²⁵ substituents;
R²⁰ is
1) NO₂,
2) CN,
3) N₃,
4) B(OR¹⁴)₂,
5) adamantyl,
6) halogen,
7) C₁-C₆ alkyl,
8) C₂-C₆ alkenyl,
9) C₂-C₄ alkynyl,
10) C₃-C₇ cycloalkyl,
11) C₃-C₇ cycloalkenyl,
12) aryl,
13) heteroaryl,
14) heterocyclyl,
15) haloalkyl,
16) OR⁷,
17) SR⁷,
18) S(O)ₙR¹⁰,
19) NR¹¹R¹²,
20) CHO,
21) C(O)R¹⁰,
22) C(O)OR¹⁴,
23) CON¹¹R¹²,
24) S(O)ₙNR¹¹R¹²,
25) NR¹⁴C(O)R¹⁰,
26) NR S(O)₂R¹⁰,
27) OC(O)R¹⁰,
28) SC(O)R¹⁰,
29) —O—(C₁-C₆alkyl-O)ₘR¹⁴, or
30) —O—(C₁-C₆ alkyl-O)ₘ—C₁-C₆ alkyl-OR¹⁴,
wherein the alkyl and the cycloalkyl are optionally substituted with one or more R¹⁵ substituents; and wherein the aryl, heteroaryl and the heterocyclyl are optionally substituted with one or more R²⁵ substituents;
R²⁵ is
1) halogen,
2) NO₂,
3) CN,
4) B(OR⁴)₂,
5) haloalkyl,
6) C₁-C₆ alkyl,
7) C₂-C₆ alkenyl,
8) C₂-C₄ alkynyl,
9) OR⁷,
10) SR⁷
11) NR¹¹R¹²,
12) CHO,
13) C(O)R¹⁰,
14) C(O)OR
15) CONR¹¹R¹²,
16) S(O)₂R¹⁰,
17) S(O)₂NR¹¹R¹².
18) NR¹⁴COR¹⁰,
19) NR¹⁴S(O)₂R¹⁰,
20) OC(O)R¹⁰, or
21) SC(O)R¹⁰;
wherein the probe is either:
a) labeled with a radioactive isotope at any suitable position;
b) linked to a detectable moiety by a suitable linker at R³ or R²⁰; or
c) linked to an affinity tag at any suitable position;
and wherein the probe binds to HSP90 protein.

10. The assay, according to claim 9, in which the probe is selected from: 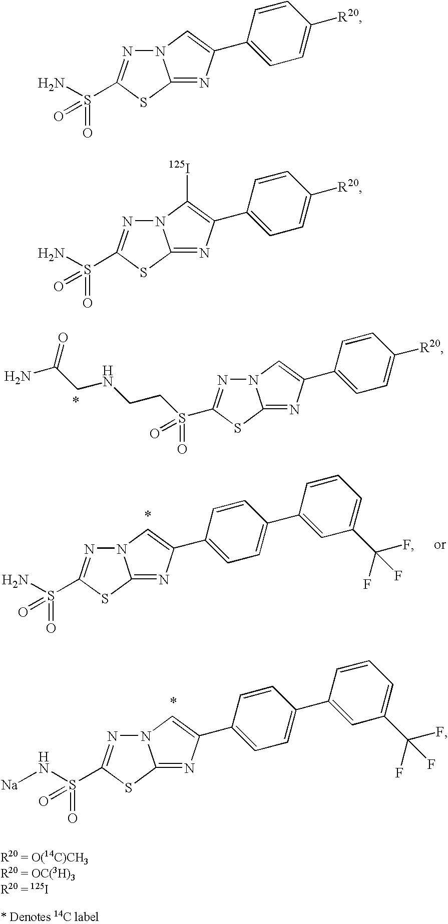
wherein R²⁰ is as defined herein.

11. The assay, according to claim 9, in which the probe is selected from:

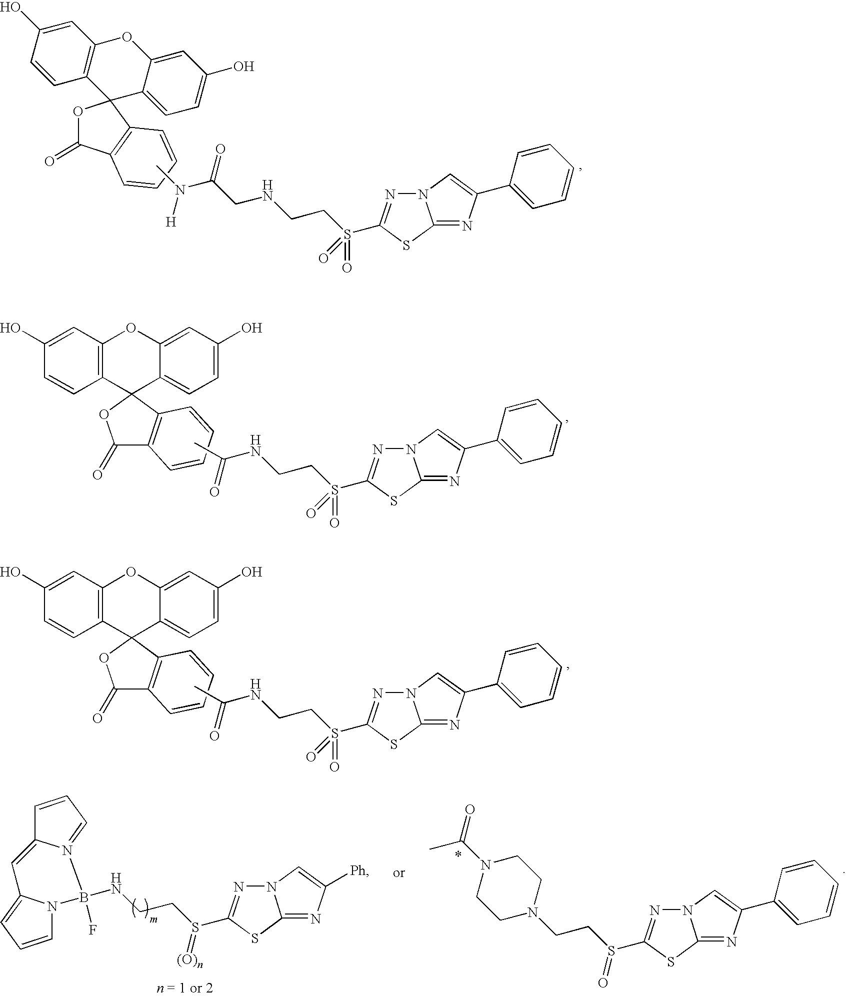

12. The assay, according to claim 1, in which the probe binds to HSP90 protein.

13. The assay, according to claim 1, in which the neuronal apoptotic pathway is an apoptotic JNK signaling pathway.

14. The assay, according to claim 1, in which the binding of the probe to the HSP90 protein causes expression of HSP70 and HSP25.

15. A probe, according to Formula I: 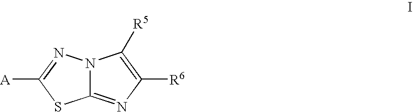
or a salt thereof,
wherein:
A is
1) —S(O)₂NR¹R²; or
2) —S(O)ₙR³;
n is 1 or 2;
m is 1 to 20;
Y is NH, O or S;
R¹ and R² are independently selected from:
1) H, or
2) C₁-C₆ alkyl;
R³ is:
1) C₁-C₆ alkyl,
2) haloalkyl,
3) aryl, or
4) heteroaryl,
wherein the alkyl is optionally substituted with one or more R¹⁵ substituents; and the aryl and heteroaryl are optionally substituted with one or more R²⁰ substituents;
R⁵ is:
1) H,
2) halogen,
3) C₁-C₆ alkyl, or
4) aryl;
R⁶ is
1) haloalkyl,
2) adamantyl,
3) aryl, or
4) heteroaryl,
wherein the aryl and the heteroaryl are optionally substituted with one or more substituents independently selected from R²⁰;
R⁷ is
1) H,
2) haloalkyl,
3) C₁-C₆ alkyl,
4) aryl,
5) heteroaryl or
6) heterocyclyl,
wherein the alkyl is optionally substituted with one or more R¹⁵ substituents; and wherein the aryl, heteroaryl and heterocyclyl is optionally substituted with one or more R²⁰ substituents;
R¹⁰ is
1) C₁-C₆ alkyl,
2) C₃-C₇ cycloalkyl,
3) haloalkyl,
4) C₂-C₆ alkenyl,
5) C₂-C₆ alkynyl,
6) C₅-C₇ cycloalkenyl,
7) aryl,
8) heteroaryl, or
9) heterocyclyl,
wherein the alkyl, cycloalkyl, alkenyl, alkynyl, cycloalkenyl are optionally substituted with one or more R¹⁵ substituents, and the aryl, heteroaryl, and heterocyclyl are optionally substituted with one or more R²⁰ substituents;
R¹¹ and R¹² are independently selected from:
1) H,
2) C₁-C₆ alkyl,
3) C₃-C₇ cycloalkyl,
4) haloalkyl,
5) aryl,
6) heteroaryl,
7) heterocyclyl,
8) C(O)—C₁-C₆ alkyl
9) C(O)—C₃-C₇ cycloalkyl
10) C(O)-aryl,
11) C(O)-heteroaryl,
12) C(O)-heterocyclyl,
13) C(O)Y—C₁-C₆ alkyl
14) C(O)Y—C₃-C₇ cycloalkyl
15) C(O)Y-aryl,
16) C(O)Y-heteroaryl,
17) C(O)Y-heterocyclyl,
wherein the alkyl and the cycloalkyl are optionally substituted with one or more R¹⁵ substituents, and the aryl, heteroaryl, and heterocyclyl are optionally substituted with one or more R²⁰ substituents;
or R¹¹ and R¹² together with the nitrogen atom to which they are bonded form a five, six or seven membered heterocyclic ring optionally substituted with one or more R²⁰ substituents;
R¹⁴ is
1) H, or
2) C₁-C₆ alkyl;
R¹⁵ is
1) NO₂,
2) CN,
3) halogen,
4) C₃-C₇ cycloalkyl,
5) haloalkyl,
6) aryl,
7) heteroaryl,
8) heterocyclyl,
9) OR⁷,
10) S(O)ₙR¹⁰,
11) NR¹¹R¹²,
12) CHO,
13) C(O)R¹⁰,
14) CO₂R¹⁴,
15) CONR¹¹R¹², or
16) S(O)ₙNR¹¹R¹²,
wherein the aryl and heteroaryl are optionally substituted with one or more R²⁵ substituents;
R²⁰ is
1) NO₂,
2) CN,
3) N₃,
4) B(OR¹⁴)₂,
5) adamantyl,
6) halogen,
7) C₁-C₆ alkyl,
8) C₂-C₆ alkenyl,
9) C₂-C₄ alkynyl,
10) C₃-C₇ cycloalkyl,
11) C₃-C₇ cycloalkenyl,
12) aryl,
13) heteroaryl,
14) heterocyclyl,
15) haloalkyl,
16) OR⁷,
17) SR⁷,
18) S(O)ₙR¹⁰,
19) NR¹¹R¹²,
20) CHO,
21) C(O)R¹⁰,
22) C(O)OR⁴,
23) CON¹¹R¹²,
24) S(O)ₙNR¹¹R¹²,
25) NR¹⁴C(O)R¹⁰,
26) NR¹⁴S(O)₂R¹⁰,
27) OC(O)R¹⁰,
28) SC(O)R¹⁰,
29) —O—(C₁-C₆alkyl-O)ₘR⁴, or
30) —O—(C₁-C₆ alkyl-O)ₘ—C₁-C₆ alkyl-OR¹⁴,
wherein the alkyl and the cycloalkyl are optionally substituted with one or more R¹⁵ substituents; and wherein the aryl, heteroaryl and the heterocyclyl are optionally substituted with one or more R²⁵ substituents;
R²⁵ is
1) halogen,
2) NO₂,
3) CN,
4) B(OR¹⁴)₂,
5) haloalkyl,
6) C₁-C₆ alkyl,
7) C₂-C₆ alkenyl,
8) C₂-C₄ alkynyl,
9) OR⁷,
10) SR⁷
11) NR¹¹R¹²
12) CHO,
13) C(O)R¹⁰,
14) C(O)OR¹⁴,
15) CONR¹¹R¹²,
16) S(O)₂R¹⁰,
17) S(O)₂NR¹¹R¹²,
18) NR¹⁴COR¹⁰,
19) NR¹⁴S(O)₂R¹⁰,
20) OC(O)R¹⁰, or
21) SC(O)R¹⁰;
wherein the probe is labeled with a detectable label or an affinity tag and wherein the probe binds to HSP90 protein.

16. The probe, according to claim 15, in which the probe is of the following formula: 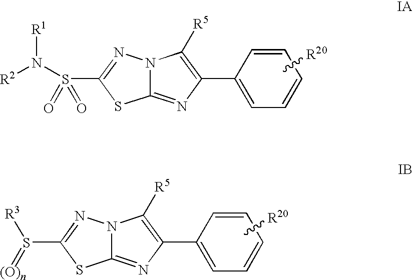
or a salt thereof,
wherein:
n is 1 or 2;
m is 1 to 20;
Y is NH, O or S;
R¹ and R² are independently selected from:
1) H, or
2) C₁-C₆ alkyl;
R³ is:
1) C₁-C₆ alkyl,
2) haloalkyl,
3) aryl, or
4) heteroaryl,
wherein the alkyl is optionally substituted with one or more R¹⁵ substituents; and the aryl and heteroaryl are optionally substituted with one or more R²⁰ substituents;
R⁵ is
1) H, or
2) halogen;
R⁷ is
1) H,
2) haloalkyl,
3) C₁-C₆ alkyl,
4) aryl,
5) heteroaryl or
6) heterocyclyl,
wherein the alkyl is optionally substituted with one or more R¹⁵ substituents; and wherein the aryl, heteroaryl and heterocyclyl is optionally substituted with one or more R²⁰ substituents;
R¹⁰ is
1) C₁-C₆ alkyl,
2) C₃-C₇ cycloalkyl,
3) haloalkyl,
4) C₂-C₆ alkenyl,
5) C₂-C₆ alkynyl,
6) C₅-C₇ cycloalkenyl,
7) aryl,
8) heteroaryl, or
9) heterocyclyl,
wherein the alkyl, cycloalkyl, alkenyl, alkynyl, cycloalkenyl are optionally substituted with one or more R¹⁵ substituents, and the aryl, heteroaryl, and heterocyclyl are optionally substituted with one or more R²⁰ substituents;
R¹¹ and R¹² are independently selected from:
1) H,
2) C₁-C₆ alkyl,
3) C₃-C₇ cycloalkyl,
4) haloalkyl,
5) aryl,
6) heteroaryl,
7) heterocyclyl,
8) C(O)—C₁-C₆ alkyl,
9) C(O)—C₃-C₇ cycloalkyl
10) C(O)-aryl,
11) C(O)-heteroaryl,
12) C(O)-heterocyclyl,
13) C(O)Y—C₁-C₆ alkyl
14) C(O)Y—C₃-C₇ cycloalkyl
15) C(O)Y-aryl,
16) C(O)Y-heteroaryl, or
17) C(O)Y-heterocyclyl,
wherein the alkyl and the cycloalkyl are optionally substituted with one or more R¹⁵ substituents, and the aryl, heteroaryl, and heterocyclyl are optionally substituted with one or more R²⁰ substituents;
or R¹¹ and R¹² together with the nitrogen atom to which they are bonded form a five, six or seven membered heterocyclic ring optionally substituted with one or more R²⁰ substituents;
R¹⁴ is
1) H, or
2) C₁-C₆ alkyl;
R¹⁵ is
1) NO₂,
2) CN,
3) halogen,
4) C₃-C₇ cycloalkyl,
5) haloalkyl,
6) aryl,
7) heteroaryl,
8) heterocyclyl,
9) OR⁷,
10) S(O)ₙR¹⁰,
11) NR¹¹R¹²,
12) CHO,
13) C(O)R¹⁰,
14) CO₂R¹⁴,
15) CONR¹¹R¹², or
16) S(O)ₙNR¹¹R¹²,
wherein the aryl and heteroaryl are optionally substituted with one or more R²⁵ substituents;
R²⁰ is
1) NO₂,
2) CN,
3) N₃,
4) B(OR¹⁴)₂,
5) adamantyl,
6) halogen,
7) C₁-C₆ alkyl,
8) C₂-C₆ alkenyl,
9) C₂-C₄ alkynyl,
10) C₃-C₇ cycloalkyl,
11) C₃-C₇ cycloalkenyl,
12) aryl,
13) heteroaryl,
14) heterocyclyl,
15) haloalkyl,
16) OR⁷,
17) SR⁷,
18) S(O)ₙR¹⁰,
19) NR¹¹R¹²,
20) CHO,
21) C(O)R¹⁰,
22) C(O)OR¹⁴,
23) CON¹¹R¹²,
24) S(O)ₙNR¹¹R¹²,
25) NR¹⁴C(O)R¹⁰,
26) NR¹⁴S(O)₂R¹⁰,
27) OC(O)R¹⁰,
28) SC(O)R¹⁰,
29) —O—(C₁-C₆alkyl-O)ₘR¹⁴, or
30) —O—(C₁-C₆ alkyl-O)ₘ—C₁-C₆ alkyl-OR¹⁴,
wherein the alkyl and the cycloalkyl are optionally substituted with one or more R¹⁵ substituents; and wherein the aryl, heteroaryl and the heterocyclyl are optionally substituted with one or more R²⁵ substituents;
R²⁵ is
1) halogen,
2) NO₂,
3) CN,
4) B(OR¹⁴)₂,
5) haloalkyl,
6) C₁-C₆ alkyl,
7) C₂-C₆ alkenyl,
8) C₂-C₄ alkynyl,
9) OR⁷,
10) SR⁷
11) NR¹¹R¹²,
12) CHO,
13) C(O)R¹⁰,
14) C(O)OR⁴,
15) CONR¹¹R¹²,
16) S(O)₂R¹⁰,
17) S(O)₂NR¹¹R¹²,
18) NR¹⁴COR¹⁰,
19) NR¹⁴S(O)₂R¹⁰,
20) OC(O)R¹⁰, or
21) SC(O)R¹⁰;
wherein the probe comprises a detectable label or an affinity tag attached to any suitable position, and wherein the probe binds to HSP90 protein and is capable of being displaced by an inhibitor apoptotic JNK signaling pathway.

17. The probe, according to claim 16, in which the probe is of the following formula: 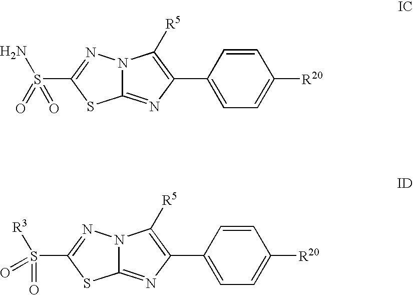
or a salt thereof,
wherein:
n is 1 or 2;
m is 1 to 20;
Y is NH, O or S;
R³ is C₁-C₆ alkyl optionally substituted with one or more R¹⁵ substituents;
R⁵ is
1) H, or
2) halogen;
R⁷ is
1) H,
2) haloalkyl,
3) C₁-C₆ alkyl,
4) aryl,
5) heteroaryl or
6) heterocyclyl,
wherein the alkyl is optionally substituted with one or more R¹⁵ substituents; and wherein the aryl, heteroaryl and heterocyclyl is optionally substituted with one or more R²⁰ substituents;
R¹⁰ is
1) C₁-C₆ alkyl,
2) C₃-C₇ cycloalkyl,
3) haloalkyl,
4) C₂-C₆ alkenyl,
5) C₂-C₆ alkynyl,
6) C₅-C₇ cycloalkenyl,
7) aryl,
8) heteroaryl, or
9) heterocyclyl,
wherein the alkyl, cycloalkyl, alkenyl, alkynyl, cycloalkenyl are optionally substituted with one or more R¹⁵ substituents, and the aryl, heteroaryl, and heterocyclyl are optionally substituted with one or more R²⁰ substituents;
R¹¹ and R¹² are independently selected from:
1) H,
2) C₁-C₆ alkyl,
3) C₃-C₇ cycloalkyl,
4) haloalkyl,
5) aryl,
6) heteroaryl,
7) heterocyclyl,
8) C(O)—C₁-C₆ alkyl,
9) C(O)—C₃-C₇ cycloalkyl
10) C(O)-aryl,
11) C(O)-heteroaryl,
12) C(O)-heterocyclyl,
13) C(O)Y—C₁-C₆ alkyl
14) C(O)Y—C₃-C₇ cycloalkyl
15) C(O)Y-aryl,
16) C(O)Y-heteroaryl, or
17) C(O)Y-heterocyclyl,
wherein the alkyl and the cycloalkyl are optionally substituted with one or more R¹⁵ substituents, and the aryl, heteroaryl, and heterocyclyl are optionally substituted with one or more R²⁰ substituents;
R¹⁴ is
1) H, or
2) C₁-C₆ alkyl;
R¹⁵ is
1) NO₂,
2) CN,
3) halogen,
4) C₃-C₇ cycloalkyl,
5) haloalkyl,
6) aryl,
7) heteroaryl,
8) heterocyclyl,
9) OR⁷,
10) S(O)ₙR¹⁰,
11) NR¹¹R¹²,
12) CHO,
13) C(O)R¹⁰,
14) CO₂R¹⁴,
15) CONR¹¹R¹², or
16) S(O)ₙNR¹¹R¹²,
wherein the aryl and heteroaryl are optionally substituted with one or more R²⁵ substituents;
R²⁰ is
1) NO₂,
2) CN,
3) N₃,
4) B(OR¹⁴)₂,
5) adamantyl,
6) halogen,
7) C₁-C₆ alkyl,
8) C₂-C₆ alkenyl,
9) C₂-C₄ alkynyl,
10) C₃-C₇ cycloalkyl,
11) C₃-C₇ cycloalkenyl,
12) aryl,
13) heteroaryl,
14) heterocyclyl,
15) haloalkyl,
16) OR⁷,
17) SR⁷,
18) S(O)ₙR¹⁰,
19) NR¹¹R¹²,
20) CHO,
21) C(O)R¹⁰,
22) C(O)OR¹⁴,
23) CONR¹¹R¹²,
24) S(O)ₙNR¹¹R¹²,
25) NR¹⁴C(O)R¹⁰,
26) NR¹⁴S(O)₂R¹⁰,
27) OC(O)R¹⁰,
28) SC(O)R¹⁰,
29) —O—(C₁-C₆alkyl-O)ₘR¹⁴, or
30) —O—(C₁-C₆ alkyl-O)ₘ—C₁-C₆ alkyl-OR¹⁴,
wherein the alkyl and the cycloalkyl are optionally substituted with one or more R¹⁵ substituents; and wherein the aryl, heteroaryl and the heterocyclyl are optionally substituted with one or more R²⁵ substituents;
R²⁵ is
1) halogen,
2) NO₂,
3) CN,
4) B(OR¹⁴)₂,
5) haloalkyl,
6) C₁-C₆ alkyl,
7) C₂-C₆ alkenyl,
8) C₂-C₄ alkynyl,
9) OR⁷,
10) SR⁷
11) NR¹¹R¹²,
12) CHO,
13) C(O)R¹⁰,
14) C(O)OR¹⁴,
15) CONR¹¹R¹²,
16) S(O)₂R¹⁰,
17) S(O)₂NR¹¹R¹²,
18) NR¹⁴COR¹⁰,
19) NR⁴S(O)₂R¹⁰,
20) OC(O)R¹⁰, or
21) SC(O)R¹⁰;
wherein the probe is either:
a) labeled with a radioactive isotope at any suitable position;
b) linked to a detectable moiety by a suitable linker at R³ or R²⁰; or
c) linked to an affinity tag at any suitable position;
and wherein the probe binds to HSP90 protein.

18. The probe, according to claim 17, in which the probe is selected from: 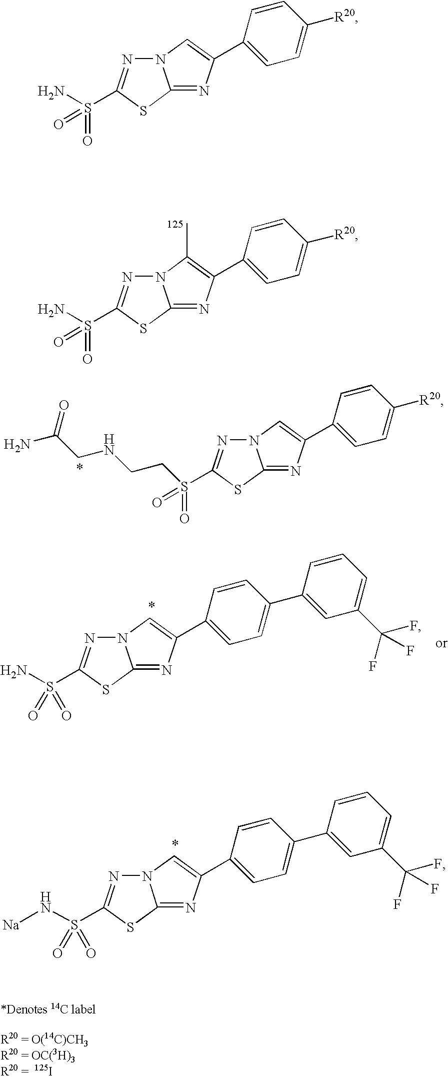
wherein R²⁰ is as defined herein.

19. The assay, according to claim 17, in which the probe is selected from:

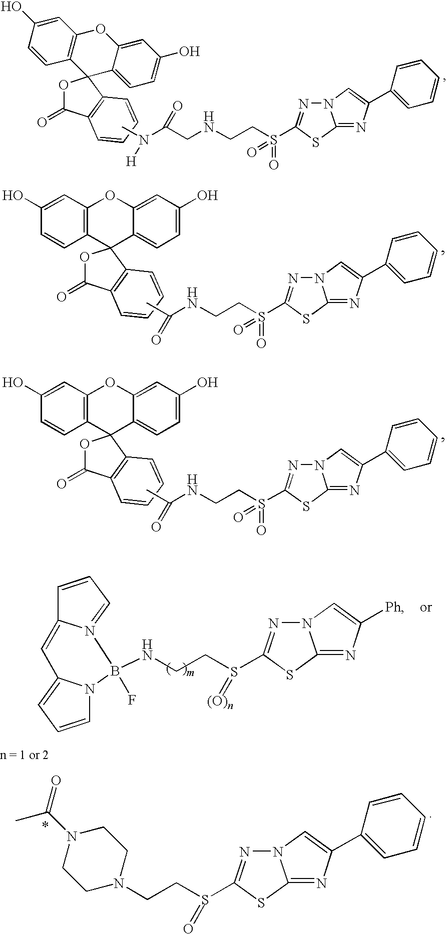

20. A probe, according to Formula I: 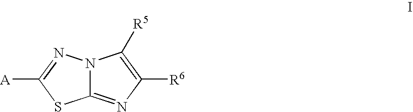
or a salt thereof,
wherein:
A is
1) —S(O)₂NR¹R²; or
2) —S(O)ₙR³;
n is 1 or 2;
m is 1 to 20;
Y is NH, O or S;
R¹ and R² are independently selected from:
1) H, or
2) C₁-C₆ alkyl;
R³ is:
1) C₁-C₆ alkyl,
2) haloalkyl,
3) aryl, or
4) heteroaryl,
wherein the alkyl is optionally substituted with one or more R¹⁵ substituents; and the aryl and heteroaryl are optionally substituted with one or more R²⁰ substituents;
R⁵ is:
1) H,
2) halogen,
3) C₁-C₆ alkyl, or
4) aryl;
R⁶ is
1) haloalkyl,
2) adamantyl,
3) aryl, or
4) heteroaryl,
wherein the aryl and the heteroaryl are optionally substituted with one or more substituents independently selected from R²⁰;
R⁷ is
1) H,
2) haloalkyl,
3) C₁-C₆ alkyl,
4) aryl,
5) heteroaryl or
6) heterocyclyl,
wherein the alkyl is optionally substituted with one or more R¹⁵ substituents; and wherein the aryl, heteroaryl and heterocyclyl is optionally substituted with one or more R²⁰ substituents;
R¹⁰ is
1) C₁-C₆ alkyl,
2) C₃-C₇ cycloalkyl,
3) haloalkyl,
4) C₂-C₆ alkenyl,
5) C₂-C₆ alkynyl,
6) C₅-C₇ cycloalkenyl,
7) aryl,
8) heteroaryl, or
9) heterocyclyl,
wherein the alkyl, cycloalkyl, alkenyl, alkynyl, cycloalkenyl are optionally substituted with one or more R¹⁵ substituents, and the aryl, heteroaryl, and heterocyclyl are optionally substituted with one or more R²⁰ substituents;
R¹¹ and R¹² are independently selected from:
1) H,
2) C₁-C₆ alkyl,
3) C₃-C₇ cycloalkyl,
4) haloalkyl,
5) aryl,
6) heteroaryl,
7) heterocyclyl,
8) C(O)—C₁-C₆ alkyl
9) C(O)—C₃-C₇ cycloalkyl
10) C(O)-aryl,
11) C(O)-heteroaryl,
12) C(O)-heterocyclyl,
13) C(O)Y—C₁-C₆ alkyl
14) C(O)Y—C₃-C₇ cycloalkyl
15) C(O)Y-aryl,
16) C(O)Y-heteroaryl,
17) C(O)Y-heterocyclyl,
wherein the alkyl and the cycloalkyl are optionally substituted with one or more R¹⁵ substituents, and the aryl, heteroaryl, and heterocyclyl are optionally substituted with one or more R²⁰ substituents;
or R¹¹ and R¹² together with the nitrogen atom to which they are bonded form a five, six or seven membered heterocyclic ring optionally substituted with one or more R²⁰ substituents;
R¹⁴ is
1) H, or
2) C₁-C₆ alkyl;
R¹⁵ is
1) NO₂,
2) CN,
3) halogen,
4) C₃-C₇ cycloalkyl,
5) haloalkyl,
6) aryl,
7) heteroaryl,
8) heterocyclyl,
9) OR⁷,
10) S(O)ₙR¹⁰,
11) NR¹¹R¹²,
12) CHO,
13) C(O)R¹⁰,
14) CO₂R¹⁴,
15) CONR¹¹R¹², or
16) S(O)ₙNR¹¹R¹²,
wherein the aryl and heteroaryl are optionally substituted with one or more R²⁵ substituents;
R²⁰ is
1) NO₂,
2) CN,
3) N₃,
4) B(OR¹⁴)₂,
5) adamantyl,
6) halogen,
7) C₁-C₆ alkyl,
8) C₂-C₆ alkenyl,
9) C₂-C₄ alkynyl,
10) C₃-C₇ cycloalkyl,
11) C₃-C₇ cycloalkenyl,
12) aryl,
13) heteroaryl,
14) heterocyclyl,
15) haloalkyl,
16) OR⁷,
17) SR⁷,
18) S(O)ₙR¹⁰,
19) NR¹¹R¹²,
20) CHO,
21) C(O)R¹⁰,
22) C(O)OR¹⁴,
23) CONR¹¹R¹²,
24) S(O)ₙNR¹¹R¹²,
25) NR¹⁴C(O)R¹⁰,
26) NR¹⁴S(O)₂R¹⁰,
27) OC(O)R¹⁰,
28) SC(O)R¹⁰,
29)-O—(C₁-C₆alkyl-O)ₘR¹⁴, or
30) —O—(C₁-C₆ alkyl-O)ₘ—C₁-C₆ alkyl-OR¹⁴,
wherein the alkyl and the cycloalkyl are optionally substituted with one or more R¹⁵ substituents; and wherein the aryl, heteroaryl and the heterocyclyl are optionally substituted with one or more R²⁵ substituents;
R²⁵ is
1) halogen,
2) NO₂,
3) CN,
4) B(OR¹⁴)₂,
5) haloalkyl,
6) C₁-C₆ alkyl,
7) C₂-C₆ alkenyl,
8) C₂-C₄ alkynyl,
9) OR⁷,
10) SR⁷
11) NR¹¹R¹²,
12) CHO,
13) C(O)R¹⁰,
14) C(O)OR¹⁴,
15) CONR¹¹R¹²,
16) S(O)₂R¹⁰,
17) S(O)₂NR¹¹R¹²,
18) NR¹⁴COR¹⁰,
19) NR¹⁴S(O)₂R¹⁰,
20) OC(O)R¹⁰, or
21) SC(O)R¹⁰;
and wherein the probe binds to HSP90 protein.

21. A probe selected from compounds 1, 2, 5, 6, 10, 11, 12, 13, 14, 15, 16, 17, 18, 19, 20, or 2-1.

22. An assay for identifying compounds that inhibit the apoptotic JNK signaling pathway, the assay comprising:
a) contacting HSP90 protein with a probe, according to claim 20, to form a probe:HSP90 complex, the probe being displaceable by a test compound;
b) measuring a signal from the probe so as to establish a reference level;
c) incubating the probe:HSP90 complex with the test compound;
d) measuring the signal from the probe;
e) comparing the signal from step d) with the reference level, a modulation of the signal indicating that the test compound binds to HSP90, and is an inhibitor of the apoptotic JNK signaling pathway.

23. An assay for identifying compounds that inhibit the apoptotic JNK signaling pathway, the assay comprising:
f) repeating steps a) to e), according to claim 22, in a high throughput screen.

24. (canceled)

25. (canceled)

26. The assay, according to claim 1, in which the signal is fluorescence, resonance energy transfer, time resolved fluorescence, radioactivity, fluorescence polarization, plasma resonance, chemiluminescence, nuclear magnetic resonance (NMR) spectroscopy, or mass spectroscopy (MS).

27. (canceled)

28. A competition binding assay for screening for compounds which bind to HSP90, the assay comprising:
a) contacting a probe, according to claim 20, with HSP90 protein to form a probe:HSP90 complex in an aqueous medium;
c) contacting the probe:HSP90 complex with at least one compound from a compound library to form a mixture;
d) measuring a STD NMR spectrum of the mixture;
e) identifying compounds which demonstrate STD or resonance shifts associated with the NMR resonances of the probe.

29. A probe comprising a compound of claim 20 which has been covalently linked to a solid support.

30. (canceled)
